(19) United States
(12) Patent Application Publication
(10) Pub. No.: US 2020/0368290 A1
(43) Pub. Date: Nov. 26, 2020

(54) METHODS AND COMPOSITIONS FOR AESTHETIC AND COSMETIC TREATMENT AND STIMULATING HAIR GROWTH

(71) Applicant: PLURISTEM LTD., Haifa (IL)

(72) Inventors: Yaacob Yanay, Shimshit (IL); Lior Raviv, Kfar Monash (IL); Murielle Agassi, Kiryat Ono (IL); Sagi Moran, Haifa (IL)

(21) Appl. No.: 16/990,838

(22) Filed: Aug. 11, 2020

Related U.S. Application Data
(63) Continuation of application No. PCT/IL2020/050363, filed on Mar. 26, 2020.
(60) Provisional application No. 62/840,457, filed on Apr. 30, 2019.
(60) Provisional application No. 62/840,478, filed on Apr. 30, 2019.
(60) Provisional application No. 62/824,826, filed on Mar. 27, 2019.
(60) Provisional application No. 62/824,856, filed on Mar. 27, 2019.
(60) Provisional application No. 62/824,790, filed on Mar. 27, 2019.

(51) Int. Cl. A61K 35/50; A61K 9/12; A61P 17/02; A61P 17/00; A61K 9/06

(52) U.S. Cl. CPC A61K 35/50; A61K 9/122; A61K 9/06; A61P 17/00; A61P 17/02

(57) ABSTRACT

Disclosed herein are methods and compositions comprising placental adherent stromal cells, conditioned media derived from a cultured placental ASC, lysates thereof, and fractions thereof, for treating a skin condition (e.g. a compromised skin barrier, acne, wrinkles, hyper/hypo-pigmentation, dryness, elastosis); increasing skin volume, and preventing or treating alopecia and related conditions.

### FIELD

Disclosed herein are methods and compositions for stimulating hair growth and aesthetic and cosmetic treatment, comprising placental-derived adherent stromal cells and factors derived therefrom.

### BACKGROUND

Skin aging is a multisystem degenerative process that involves the skin and the skin support system (Sjerobabski & Poduje, 2008). The process of skin aging may be divided into intrinsic and extrinsic aging. It may be caused by several factors, such as, UV irradiation, stress, ROS generation or smoking. Wrinkle formation characterizes photo-aged skin and can be caused by degradation of collagen fibrils and gelatin fibers. Further, because of increased melanin synthesis, hyper-pigmented skin is observed in various dermatological disorders, namely melasma, solar lentigines and ephelides. These clinical conditions are due to frequent exposure to UV rays and certain drugs and chemicals, resulting in skin darkening. Depigmenting agents commonly are prescribed to treat such disorders. Commercially available skin lightening and depigmentation agents include magnesium-1-ascorbyl-2-phosphate (MAP), hydroxyanisole, N-acetyl-4-S-cysteaminylphenol, arbutin (hydroquinone-beta-d-glucopyranoside) and hydroquinone (HQ) (Parvez S et al, 2006). Some adverse effects of these synthetic compounds are irreversible cutaneous damage, ochronosis etc. These adverse effects have led to the search for alternative cosmetic formulations.

Compromised Skin Barrier

Transepidermal water loss (TEWL) is a term used in dermatology to characterize the loss of water that passes from the inside of a body through the epidermal layer (skin) to the surrounding atmosphere via diffusion and evaporation processes. TWEL is also used to assess compromised skin barrier function.

TEWL can have genetic and/or environmental etiology. It can be the result of a genetic polymorphism leading to a decrease in protective protein expression and thus compromised skin barrier. Skin inflammation, mainly caused by an external irritant, can also lead to water loss. Both genetic and environmental components can together or separately lead to excessive TEWL and ultimately trigger different TEWL-associated skin diseases that range from dry skin to more severe conditions such as eczema.

TEWL can cause dry skin or reactive skin or eczema. In some instances, for example when linked to the exposure to an allergen through the skin, this can lead to an allergic eczema/atopic dermatitis, i.e. an eczema accompanied by allergic sensitization.

In TEWL-associated disorders, the normal water loss rate is increased due to a diminished barrier function of the epidermis, causing dehydrated epidermis, which sometimes manifests as irritation and/or dry or scaly skin and is often associated with atopic dermatitis (a.k.a. eczema) reactive skin (e.g., winter rashes) and/or vulnerability to infections. Other diseases that increase TEWL and skin inflammation include chronological aging. Increased TEWL may also be secondary to injury, infection, burns, psoriasis, and inflammatory skin conditions such as atopic diathesis in rosacea and perioral dermatitis.

Alopecia

There are a number of types of alopecia, including androgenic alopecia (also referred to as male or female pattern hair loss), acute alopecia, and alopecia areata including alopecia totalis and alopecia universalis.

Androgenic alopecia is the most common form of alopecia. Androgenic alopecia is a hereditary hair-loss condition affecting men and women of, for example, Caucasian or Asian descent. Androgenic alopecia is characterized by a progressive decrease in hair volume, or even baldness. Without treatment, the number of hairs on a sufferer of androgenic alopecia will decrease at a rate of approximately 5% per year after onset e.g., Ellis et al, Expert Reviews in Molecular Medicine, 4:1-11, 2002. Androgenic alopecia is reported to affect up to 70% of the general population, with an estimated 30% of men developing androgenic alopecia by the age of 30, and 50% of men affected by the age of 50 (Sinclair R, JMHG, 1(4):319-327, 2004; Lee and Lee, Ann. Dermatol., 24(3):243-252, 2012). As many as 10% of pre-menopausal women are reported to exhibit signs of female pattern hair loss, and the incidence increases significantly as women enter menopause, affecting as many as 50-75% of women aged 65 years or older (Norwood O T, Dermatol Surg., 27(1):53-4, 2001).

### SUMMARY

Provided herein are methods and compositions for aesthetic, cosmetic, and beauty treatments and stimulating hair growth, comprising placental adherent stromal cells, their lysates or conditioned media, or fractions derived therefrom.

Placental adherent stromal cells (ASC) refers to adherent stromal cells from placental tissue. Conditioned medi[a]/[um]/CM, as used herein, refers to a growth medium that has been used to incubate a cell culture. The present disclosure is not intended to be limited to particular medium formulations; rather, any medium suitable for incubation of placental ASC is encompassed. Reference herein to “cultured” placental ASC refers to ASC expanded according to the methods mentioned herein, each of which represents a separate embodiment.

In certain embodiments, the described placental ASC have been cultured on a 2-dimensional (2D) substrate, a 3-dimensional (3D) substrate, or a combination thereof. Non-limiting examples of 2D and 3D culture conditions are provided in the Detailed Description and in the Examples.

Alternatively or in addition, the placental ASC are allogeneic to the subject; or, in other embodiments, are autologous; or, in other embodiments, are xenogeneic

Reference herein to “growth” of a population of cells is intended to be synonymous with expansion of a cell population. In certain embodiments, ASC (which may be, in certain embodiments, placental ASC), are expanded without substantial differentiation. In various embodiments, the described expansion is on a 2D substrate, on a 3D substrate, or a 2D substrate, followed by a 3D substrate.

Except where otherwise indicated, all ranges mentioned herein are inclusive.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the embodiments of the invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a diagram of a bioreactor that can be used to prepare the cells.

FIG. 2 contains pictures of bone marrow (BM)-derived MSC (top row) or placental cells after adipogenesis assays. Cells were incubated with (left column) or without (right column) differentiation medium. Placental ASC were expanded in SRM (middle 3 rows depict 3 different batches) or in full DMEM (bottom row).

FIG. 3 contains pictures of BM-derived MSC (top row) or placental cells after osteogenesis assays. Cells were incubated with (left column) or without (right column) differentiation medium. Placental ASC were expanded in SRM (middle 3 rows depict 3 different batches) or in full DMEM (bottom row).

FIG. 4. Illustration of StageTips apparatus.

FIGS. 5A-5J are plots showing luminescence of Luminex® beads, reflective of concentration (vertical axis), for IL-1-ra, Collagen IV-la, Fibronectin, IL-13, HGF, VEGF-A, IL-4, PDGF-AA, TIMP-1, TGFb2, and TGFb1 (in A-J, respectively). P250416 R21 and P150518 R02 are maternal batches; R090418 RO1 and R170216 R19 are fetal/serum batches; and PD060918S2 437BR01; PD030316 441BR09 are fetal SF batches. Bioreactor CM from various batches (horizontal axis) were subjected to no treatment (BR; lanes 1-6 from left), Tangential Flow

Filtration (TFF; Pall Corporation; lanes 7-12), or lyophilization (LYP; lanes 13-18) upper panels. Lower panels depict analyses of conditioned medium generated in plates, with a higher cell/medium ratio.

FIGS. 6A-6B are plots showing expression of angiogenetic factors (horizontal axis), as assessed by Luminex® (A) or ELISA (B). Expression, measured by fluorescence intensity, is shown on the vertical axis. ASC were incubated under normal or hypoxic conditions (left and right bar in each series)

FIG. 7 is a plot of fibroblast population doubling (vertical axis) after 72 hours in culture, in growth medium (lanes 1, 3, 5, and 7) or medium mixed with resuspended ASC-CM (lanes 2, 4, 6, and 8). Lanes 1-2, 3-4, 5-6, and 7-8 depict fibroblasts aged 0, 2.1, 8.6, and 12.3 PD, respectively.

FIG. 8 is a plot of fibroblast viability (vertical axis; expressed as percentage of viable cells of the number of cells immediately after exposure to H₂O₂) following exposure to H₂O₂ and incubation with growth media (solid line) or ASC-CM (dotted line).

FIGS. 9A-9B are plots of migration of young (A) and old (B) fibroblasts, as assessed by cell density in the wound area (vertical axis) in a scratch wound assay, in SF-DMEM (lighter line) or fetal placental ASC-CM lyophilized and resuspended in SF DMEM (darker line). FIGS. 9C-9D are plots of migration of young (C) and old (D) fibroblasts, assessed and plotted in the same manner, in SF-DMEM (lighter line) or straight fetal placental ASC-CM (darker line).

FIG. 10 is a plot of DP population doubling (vertical axis) after 72 hours in culture, in growth medium (lanes 1, 3, 5, and 7) or medium mixed with resuspended ASC-CM (lanes 2, 4, 6, and 8). Lanes 1-2, 3-4, 5-6, and 7-8 depict fibroblasts aged 0, 2.1, 8.6, and 12.3 PD, respectively.

FIGS. 11A-11C are plots showing blood flow (A; vertical axis), or formation of functional new blood vessels (vertical axis) of 1-4 (B) or 4-8 (C) micron diameter. CD34 staining indicates new blood vessels, and FITC Dextran indicates blood vessel functionality. In A, upper solid and dotted lines show data from animals treated in the operated and contralateral legs; lower dark and gray lines show animals given placebo treatment of operated and contralateral legs. In B-C, the 1^{st} and 2^{nd} bar in each series show animals given placebo- and ASC-treatment in the operated limb.

FIG. 12 shows photographs affected toe of a patient with Buerger's disease before (left panel) and after (right panel) treatment.

### DETAILED DESCRIPTION

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Aspects of the invention relate to methods and compositions that comprise placental adherent stromal cells (ASC), their lysates or conditioned media, and fractions derived therefrom. In some embodiments, the ASC may be human ASC, or in other embodiments animal ASC.

In one embodiment, there is provided a method for treating, or in another embodiment preventing, or in another embodiment ameliorating, a skin condition in a subject, comprising administering a composition that comprises cultured placental ASC, thereby treating, preventing, or lessening the severity of a skin condition. As provided herein, effective amounts of the described compositions ameliorate various skin conditions. In various embodiments, the placental ASC are maternal tissue-derived ASC (ASC from a maternal portion of the placenta); fetal tissue-derived ASC (ASC from a fetal portion of the placenta); or a mixture thereof. Alternatively or in addition, the placental ASC are allogeneic to the subject; or, in other embodiments, are autologous; or, in other embodiments, are xenogeneic. In certain embodiments, the composition is an injected composition.

In one embodiment, there is provided a method for treating, or in another embodiment preventing, or in another embodiment ameliorating, a skin condition in a subject, comprising administering a composition that comprises a conditioned medium (CM) of cultured placental ASC, their lysates or conditioned media, or fractions derived therefrom, thereby treating, preventing, or lessening the severity of a skin condition. In various embodiments, the placental ASC are maternal tissue-derived ASC (ASC from a maternal portion of the placenta); fetal tissue-derived ASC (ASC from a fetal portion of the placenta); or a mixture thereof. Alternatively or in addition, the placental ASC are allogeneic to the subject; or, in other embodiments, are autologous; or, in other embodiments, are xenogeneic. In certain embodiments, the composition is an injected composition.

In another embodiment, there is provided a composition for treating, preventing, or ameliorating a skin condition in a subject, comprising cultured placental ASC, their lysates or conditioned media, or fractions derived therefrom. In certain embodiments, the composition is an injected composition. In various embodiments, the placental ASC are maternal tissue-derived ASC; fetal tissue-derived ASC; or a mixture thereof. Alternatively or in addition, the placental ASC are allogeneic to the subject; or, in other embodiments, are autologous. Placental ASC, lysates and CM thereof, and fractions derived therefrom each represents a separate embodiment.

The described skin condition may be, in various embodiments, a side effect of a facial treatment, non-limiting examples of which are laser resurfacing and chemical peel treatment. A more specific embodiment of the side effect is a compromised skin barrier. In other embodiments, the condition is a post micro-needling treatment side effect, a mesotherapy side effect; acne; wrinkle formation; skin aging (a more specific example of which is skin photoaging); reduced skin elasticity; skin lacerations; a hyperpigmentation blemish; a hypopigmentation blemish; skin dryness; thinning of the epidermis; or an elastosis. In still other embodiments, the skin condition is atopic dermatitis. Each condition represents a separate embodiment. Enhancing regeneration of skin from various injuries, including, in some embodiments, those enumerated herein, is a further embodiment. Laser resurfacing, in various embodiments, may be ablative or non-ablative.

Chemical peel, as used herein, refers to a technique used to improve the appearance of the skin on the face, neck or hands. A chemical solution is applied to the skin that causes it to exfoliate and eventually peel off, resulting regenerated skin that is usually smoother and less wrinkled.

In other embodiments, there is provided a method for reducing transepidermal water loss (TEWL) in a subject, comprising administering a composition that comprises cultured placental ASC, their lysates or conditioned media, or fractions derived therefrom, thereby reducing TEWL. As provided herein, the described ASC secrete factors such as SOD1 and SOD2 (superoxide dismutase 1 and 2; Uniprot Nos. P00441 and P04179, respectively), which play roles in skin barrier integrity, and they stimulation proliferation of dermal fibroblasts and protect them from oxidative damage. In various embodiments, the placental ASC are maternal tissue-derived ASC; fetal tissue-derived ASC; or a mixture thereof. Alternatively or in addition, the placental ASC are allogeneic to the subject; or, in other embodiments, are autologous; or, in other embodiments, are xenogenic. In certain embodiments, the composition is an injected composition. In certain embodiments, the TEWL is secondary to injury, infection, a burn, atopic dermatitis, or psoriasis. Placental ASC, lysates and CM thereof, and fractions derived therefrom each represents a separate embodiment.

In still other embodiments, there is provided a composition for reducing TEWL in a subject, comprising a composition that comprises cultured placental ASC, their lysates or conditioned media, or fractions derived therefrom. In certain embodiments, the composition is an injected composition. In various embodiments, the placental ASC are maternal tissue-derived ASC; fetal tissue-derived ASC; or a mixture thereof. Alternatively or in addition, the placental ASC are allogeneic to the subject; or, in other embodiments, are autologous. In certain embodiments, the TEWL is secondary to injury, infection, a burn, atopic dermatitis, or psoriasis. Placental ASC, lysates and CM thereof, and fractions derived therefrom each represents a separate embodiment.

In other embodiments, there is provided a method for reducing, or in another embodiment ameliorating, skin inflammation in a subject, comprising administering a composition that comprises cultured placental ASC, their lysates or conditioned media, or fractions derived therefrom, thereby reducing or ameliorating skin inflammation. As provided herein, effective amounts of the described compositions reduce skin inflammation. In various embodiments, the placental ASC are maternal tissue-derived ASC; fetal tissue-derived ASC; or a mixture thereof. Alternatively or in addition, the placental ASC are allogeneic to the subject; or, in other embodiments, are autologous; or, in other embodiments, are xenogenic. In certain embodiments, the composition is an injected composition. In certain embodiments, the skin inflammation is secondary to atopic diathesis in rosacea, or, in other embodiments, perioral dermatitis. Placental ASC, lysates and CM thereof, and fractions derived therefrom each represents a separate embodiment.

In still other embodiments, there is provided a composition for reducing or ameliorating skin inflammation in a subject, comprising cultured placental ASC, their lysates or conditioned media, or fractions derived therefrom. In certain embodiments, the composition is an injected composition. In various embodiments, the placental ASC are maternal tissue-derived ASC; fetal tissue-derived ASC; or a mixture thereof. Alternatively or in addition, the placental ASC are allogeneic to the subject; or, in other embodiments, are autologous. In certain embodiments, the skin inflammation is secondary to atopic diathesis in rosacea, or, in other embodiments, perioral dermatitis. Placental ASC, lysates and CM thereof, and fractions derived therefrom each represents a separate embodiment.

In still other embodiments, there is provided a method for treating, or in other embodiments preventing, or in other embodiments ameliorating hair loss in a subject, comprising administering a composition that comprises cultured placental ASC (or, in other embodiments, a population of cultured placental ASC), thereby treating, preventing, or lessening the severity of hair loss. In certain embodiments, the composition is a topical composition. In certain embodiments, the composition is a gel. In other embodiments, the composition is a lotion. In still other embodiments, the composition is a foam. In yet other embodiments, the composition is an aqueous solution, or, in other embodiments, a suspension. In other embodiments, the composition is a shampoo comprising a CM, lysate, or fraction derived from placental ASC. In other embodiments, the composition is an injectable formulation. As provided herein, effective amounts of the described compositions ameliorate hair loss, and, in other embodiments, augment hair growth. In various embodiments, the placental ASC are maternal tissue-derived ASC; fetal tissue-derived ASC; or a mixture thereof. Alternatively or in addition, the placental ASC are allogeneic to the subject; or, in other embodiments, are autologous. Placental ASC, lysates and CM thereof, and fractions derived therefrom each represents a separate embodiment. As provided herein, the described ASC secrete HGF, PDGF, MMP-2, and/or VEGF, which play roles in hair follicle health, and they stimulation replication of dermal papilla cells.

In still other embodiments, there is provided a method for treating, or in other embodiments preventing, or in other embodiments ameliorating hair loss in a subject, comprising administering a composition that comprises a CM or lysate of a cultured placental ASC (or, in other embodiments, a population of cultured placental ASC), or, in other embodiments, a fraction derived from the CM. thereby treating, preventing, or lessening the severity of hair loss. In certain embodiments, the composition is a topical composition. In certain embodiments, the composition is a gel. In other embodiments, the composition is a lotion. In still other embodiments, the composition is a foam. In yet other embodiments, the composition is an aqueous solution, or, in other embodiments, a suspension. In other embodiments, the composition is a shampoo comprising a medium, lysate, or fraction derived from placental ASC. In other embodiments, the composition is an injectable formulation. As provided herein, effective amounts of the described compositions ameliorate hair loss, and, in other embodiments, augment hair growth. In various embodiments, the placental ASC are maternal tissue-derived ASC; fetal tissue-derived ASC; or a mixture thereof. Alternatively or in addition, the placental ASC are allogeneic to the subject; or, in other embodiments, are autologous. Placental ASC lysates, ASC-CM, and fractions derived therefrom each represents a separate embodiment.

In still other embodiments, there is provided a composition for treating, preventing, or ameliorating hair loss in a subject, comprising cultured placental ASC, their lysates or CM, or fractions derived therefrom. In certain embodiments, the composition is a gel. In other embodiments, the composition is a lotion. In still other embodiments, the composition is a foam. In yet other embodiments, the composition is an aqueous solution, or, in other embodiments, a suspension, which may be, in some embodiments, an injectable formulation. In various embodiments, the placental ASC are maternal tissue-derived ASC; fetal tissue-derived ASC; or a mixture thereof. Alternatively or in addition, the placental ASC are allogeneic to the subject; or, in other embodiments, are autologous. Placental ASC, lysates and CM thereof, and fractions derived therefrom each represents a separate embodiment.

In still other embodiments, there is provided a method for treating, or in other embodiments preventing, or in other embodiments ameliorating alopecia in a subject, comprising administering a topical composition that comprises cultured placental ASC, their lysates or CM, or fractions derived therefrom, thereby treating, preventing, or lessening the severity of alopecia. In certain embodiments, the composition is a gel. In other embodiments, the composition is a lotion. In still other embodiments, the composition is a foam. In yet other embodiments, the composition is an aqueous solution, or, in other embodiments, a suspension. In other embodiments, the composition is a shampoo comprising a CM, lysate, or fraction derived from placental ASC. In other embodiments, the composition is an injectable formulation. As provided herein, effective amounts of the described compositions ameliorate alopecia. In various embodiments, the placental ASC are maternal tissue-derived ASC; fetal tissue-derived ASC; or a mixture thereof. Alternatively or in addition, the placental ASC are allogeneic to the subject; or, in other embodiments, are autologous. In some embodiments, the alopecia is not mediated by auto-immunity. In some embodiments, the alopecia is not mediated by auto-immunity. In certain embodiments, the alopecia is androgenic alopecia, which may be, in various embodiments, male or female androgenic alopecia. Methods of assessing hair regeneration in animal models are known in the art, and are described, for example, in Bak D H et al. and the references cited therein, Methods of assessing hair regeneration in cell culture models are known in the art, and are described, for example, in Madaan A et al., Rajendran R L et al., Hwang I et al., and the references cited therein. Placental ASC, lysates and CM thereof, and fractions derived therefrom each represents a separate embodiment.

In still other embodiments, there is provided a topical composition for treating, preventing, or ameliorating alopecia in a subject, comprising cultured placental ASC, their lysates or conditioned media, or fractions derived therefrom. In certain embodiments, the composition is a gel. In other embodiments, the composition is a lotion. In still other embodiments, the composition is a foam. In yet other embodiments, the composition is an aqueous solution, or, in other embodiments, a suspension. In various embodiments, the placental ASC are maternal tissue-derived ASC; fetal tissue-derived ASC; or a mixture thereof. Placental ASC, lysates and CM thereof, and fractions derived therefrom each represents a separate embodiment.

In other embodiments, there is provided a method of improving skin tone, comprising administration of a cultured placental ASC (or, in other embodiments, a population of cultured placental ASC), their lysates or CM, or fractions derived therefrom. Methods of treating skin with ASC-derived factors are known in the art, and are described, for example, in Kim E S et al. and the references cited therein. Placental ASC, lysates and CM thereof, and fractions derived therefrom each represents a separate embodiment.

In yet other embodiments, there is a provided a method for increasing a volume under a skin of a subject, comprising injecting a filler composition, the composition comprising cultured placental ASC, their lysates or CM, or fractions derived therefrom, thereby increasing a volume under a skin. As provided herein, effective amounts of the described compositions increase the volume under the skin of a subject; or in other embodiments increase the skin volume of a subject. In various embodiments, the placental ASC are maternal tissue-derived ASC; fetal tissue-derived ASC; or a mixture thereof. In certain embodiments, the placental ASC are alive. Alternatively or in addition, the placental ASC are allogeneic to the subject; or, in other embodiments, are autologous. In some embodiments, the herein-described filler compositions are injectable filler compositions. Placental ASC, lysates and CM thereof, and fractions derived therefrom each represents a separate embodiment.

In still other embodiments, there is provided an injectable filler composition, comprising cultured placental ASC, their lysates or CM, or fractions derived therefrom. In various embodiments, the placental ASC are maternal tissue-derived ASC; fetal tissue-derived ASC; or a mixture thereof. Alternatively or in addition, the placental ASC are allogeneic to the subject; or, in other embodiments, are autologous. Placental ASC, lysates and CM thereof, and fractions derived therefrom each represents a separate embodiment.

In certain embodiments, the described filler methods and compositions are targeted to a skin area that is deficient in volume. Those skilled in the art will appreciate that such areas can be identified by a beautician.

Alternatively or in addition, the described filler composition comprises a suspension of placental ASC; which may be present, in further embodiments, in combination with a semi-solid or gel carrier composition. In other embodiments, the filler composition comprises placental ASC that have been seeded on a scaffold. In other embodiments, the filler composition further comprises substances that enhance the activity of filler compositions, a non-limiting example of which is hyaluronic acid.

Cell Lysates

In certain embodiments, the therapeutic agent is a lysate that is derived from a cultured placental ASC. “Lysate”, as used herein, refers to a composition produced after subjecting a cell population with an agent that disrupts the cell membrane. Wherever reference is made herein to a cultured placental ASC, a population of cultured placental ASC can be used, in other embodiments.

Fractions of CM and Cell Lysates

As mentioned, the described methods and compositions comprise, in certain embodiments, a fraction of a placental ASC-CM. In other embodiments, the methods and compositions comprise a fraction of a placental ASC lysate. Each possibility represents a separate embodiment, and each may be freely combined with each fractionation methodology.

The described fraction is, in certain embodiments, a CM of a placental ASC (“placental ASC-CM”), or, in other embodiments, of a placental ASC lysate. The term placental ASC-CM, except where indicated otherwise, refers to a growth medium in which placental ASC were incubated. In certain embodiments, the CM was subsequently separated from the ASC. In more specific embodiments, placental ASC were incubated in the CM under conditions compatible with cell growth, for 6-150 hours; or, in other embodiments, for 6-144 hours; 6-120 hours; 6-96 hours; 6-72 hours; 6-48 hours; 6-36 hours; 6-24 hours; 12-150 hours; 12-144 hours; 12-120 hours; 12-96 hours; 12-72 hours; 12-48 hours; 12-36 hours; 12-24 hours; 24-150 hours; 24-144 hours; 24-120 hours; 24-96 hours; 24-72 hours; 24-48 hours; or 24-36 hours.

In other embodiments, the described placental ASC lysate or ASC-CM is subjected to lyophilization, which may be, in more specific embodiments, freeze drying or spray drying. In certain embodiments, the lyophilizate is subjected to encapsulation, and/or, in other embodiments, incorporated into an emulsion. Each embodiment of lyophilization, encapsulation, and emulsions may be freely combined.

In still other embodiments, the placental ASC lysate or ASC-CM is subjected to dialysis. In more specific embodiments. In more specific embodiments, the dialysis membrane may have a cutoff value of 2-50 Kda (kilodaltons). In other embodiments, the cutoff is 2-100, 2-70, 2-40, 2-30, 2-20, 2-15, 2-10, 3-100, 3-70, 3-40, 3-30, 3-20, 3-15, 3-10, 5-100, 5-70, 5-40, 5-30, 5-20, 5-15, 5-10, 7-100, 7-70, 7-40, 7-30, 7-20, 7-15, 7-10, 10-100, 10-70, 10-40, 10-30, 10-20, or 10-15 kDa. In certain embodiments, the dialysate is subjected to encapsulation, and/or, in other embodiments, incorporated into an emulsion. Each embodiment of dialysis, encapsulation, and emulsions may be freely combined.

In more specific embodiments, the fraction may be enriched in secreted proteins. Non-limiting examples of fractions enriched in secreted proteins are protein extracts.

In other embodiments, the fraction is enriched in peptides. Peptide, as used herein, refers to protein or protein fragment not more than 50 amino acid residues in length.

In still other embodiments, the fraction is enriched in secreted lipids.

In still other embodiments, the vesicular component is enriched in extracellular vesicles, which may be exosomes; or, in other embodiments, microvesicles; or, in other embodiments, exomeres. In yet other embodiments, the vesicular component consists essentially of extracellular vesicles, which may be exosomes; or, in other embodiments, microvesicles; or, in other embodiments, exomeres. In still other embodiments, the vesicular component consists of extracellular vesicles, which may be exosomes, or, in other embodiments, microvesicles. In still other embodiments, the vesicular component comprises extracellular vesicles, which may be exosomes; or, in other embodiments, microvesicles; or, in other embodiments, exomeres.

Microvesicles (also referred to herein as microparticles) are, in various embodiments, identified based on their size (e.g. 100 nm to 1 μm), surface markers, or the exposure of the negatively charged phosphatidylserine in the outer membrane (Johnstone et al and Pan et al). Methods for isolating microvesicles are known in the art and are described, for example, in Hugel et al and VanWijk et al).

Exomeres, in certain embodiments, refers to nonmembranous secreted nanoparticles, which average about 35 nm (nanometers) in size. In other embodiments, the exomeres are secreted nanoparticles that have a size smaller than 50 nm (e.g. 1-50 nm) and a stiffness in the range of 140-820 megapascals (Mpa). Exomeres are described in Zhang H et al.

In certain embodiments, the described methods comprise isolation of microparticles by centrifugation and optional flow cytometry, for example as described in Burger D et al or the references cited therein. One such protocol, provided solely for purposes of exemplification, involves a low-speed centrifugation to remove large cellular debris, fluorescent labeling of surface proteins, and cytometry-based sorting. The low-speed centrifugation can e.g. be for 15 minutes at 1500×g. In certain embodiments, the supernatant from this centrifugation is pelleted again, to ensure removal of large debris. Microparticles can then be pelleted, e.g. by centrifugation at 20,000×g for 20 minutes. The pellet is then resuspended and then, to obtain a high-purity preparation, may be stained with a microparticle surface marker (e.g. Annexin) and subjected to flow cytometry. An upper size limit (e.g. 1 micron) may be established using the forward scatter and side scatter parameters, as will be understood by those skilled in the art of flow cytometry.

In other embodiments, the methods comprise isolation of microparticles by centrifugation, for example as described in Braga-Lagache et al, or the references cited therein. One such protocol, provided solely for purposes of exemplification, involves a pre-clearing centrifugation step for 2 min at 16,000×g at RT. The supernatant is then centrifuged at 16000×g and RT for 20-40 min. The supernatant is then aspirated, and the pellets are reconstituted in buffered solution. MP are optionally pelleted again by centrifugation at 16,000×g and RT for 20 min, followed by 1-2 more optional washing steps.

In yet other embodiments, the fraction is enriched in exosomes (e.g. by ultracentrifugation). In more specific embodiment, the fraction may include exosome stabilizing agents. Methods for preparing exosomes are known in the art, and are described, for example in Mincheva-Nilsson L et al. (Isolation and Characterization of Exosomes from Cultures of Tissue Explants and Cell Lines. Curr Protoc Immunol. 2016 Nov. 1; 115:14.42.1-14.42.21); Al-Nedawi K et al. (Analysis of Extracellular Vesicles in the Tumor Microenvironment. Methods Mol Biol. 2016; 1458:195-202. doi: 10.1007/978-1-4939-3801-8_14); Pin Li et al (Progress in Exosome Isolation Techniques. Theranostics. 2017; 7(3): 789-804. doi: 10.7150/thno.18133); and Ban J J et al. (Low pH increases the yield of exosome isolation. Biochem Biophys Res Commun. 2015 May 22; 461(1):76-9. doi: 10.1016/j.bbrc.2015.03.172). In still other embodiments, exosomes can be isolated by first pre-clearing media to remove cells, centrifugation for 30 min at 15,000 g to remove cellular debris, and pelleting of exosomes from the supernatant by ultracentrifugation at 150,000 g for 90 min (Jethwa S A et al., Exosomes bind to autotaxin and act as a physiological delivery mechanism to stimulate LPA receptor signalling in cells. J Cell Sci. 2016 Oct. 15; 129(20):3948-3957).

In yet other embodiments, the fraction is a soluble fraction. In other embodiments, the fraction is a pelletable fraction. Non-limiting examples of methods for preparing solid and pelletable fractions are described in Bach F C et al. (Soluble and pelletable factors in porcine, canine and human notochordal cell-conditioned medium: implications for IVD regeneration. Eur Cell Mater. 2016 Aug. 30; 32:163-80).

In yet other embodiments, the fraction is produced using size exclusion chromatography (e.g. Sephadex™ columns)

Methods for fractionating CM and cell lysates are known in the art, and are described, for example in the product literature for the GELFREE® 8100 Fractionation System (Expedeon, San Diego, Calif.), which enables preparative-scale fractionation of analytes according to electrophoretic mobility; and Weng Y et al. (In-Depth Proteomic Quantification of Cell Secretome in Serum-Containing Conditioned Medium. Anal Chem. 2016 May 3; 88(9):4971-8. doi: 10.1021/acs.analchem.6b00910).

In other embodiments, the fraction is produced using an aqueous two-phase system (ATPS). Such systems are known in the art, and are described, for example, in Mujahid Iqbal et al. (Aqueous two-phase system (ATPS): an overview and advances in its applications. Biol Proced Online. 2016; 18: 18. doi: 10.1186/s12575-016-0048-8). In certain, more specific embodiments, the system is a biphasic system formed by two polymers (which are, in certain embodiments, polyethylene glycol [PEG] and dextran). In other embodiments, the system is formed by a polymer and a salt (non-limiting embodiments of which are phosphate, sulfate or citrate). In other embodiments, ionic liquids and short-chain alcohols are utilized (Grilo A L et al; Van Berlo M et al). In other embodiments, ionic and/or non-ionic surfactants are used for the formation of micellar and reverse micellar ATPSs (Liu C et al; and Xiao J X et al). In yet other embodiments, the system is a polymer/polymer system or, in other embodiments, a polymer/salt systems (Albertsson P Å). In yet other embodiments the system is an alcohol-salt ATPS (Louwrier A; and Jiang B et al.). In still other embodiments, the system is an aqueous micellar two-phase system (Bordier C. 1981); a mixed micellar system (Lye G J et al); an ionic liquids (ILs)-based ATPS (Berthod A et al.); or a poly-phase system (e.g. with three or four polymer phases) also have been constructed for the separation of biomolecules (Hatti-Kaul R 2001).

In other embodiments, the system is a one-polymer ATPS, which utilizes only one polymer for the formation of ATPS in water (Johansson H-O et al).

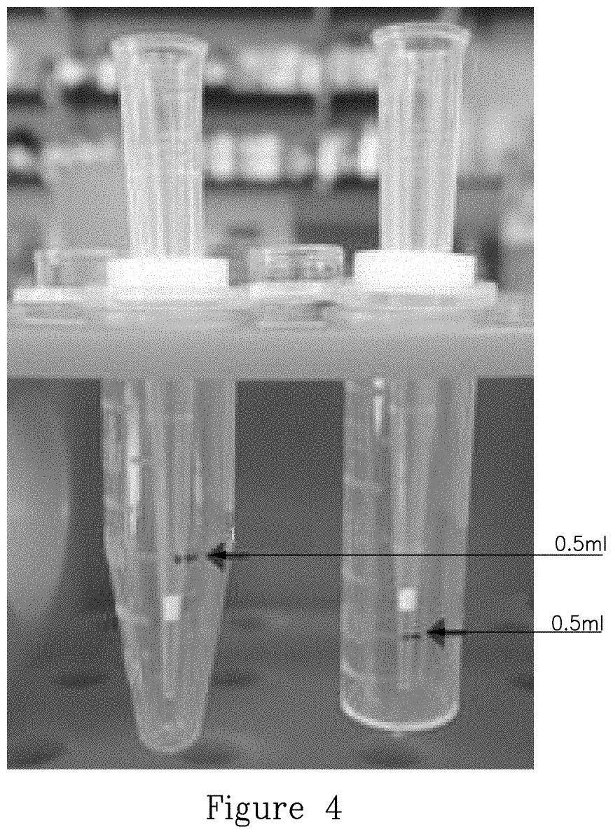

By way of exemplification, StageTips (FIG. 4) may be used in the described methods and compositions (Yanbao Yu et al., A spinnable and automatable StageTip for high throughput peptide desalting and proteomics. Protocol Exchange (2014) doi:10.1038/protex.2014.033; Rappsilber J et al., Protocol for micro-purification, enrichment, pre-fractionation and storage of peptides for proteomics using StageTips. Nat Protoc. 2007; 2(8):1896-906).

A non-limiting StageTip protocol, provided for exemplification purposes only, utilizes the following buffers and reagents:
Buffer A: 100% methanol; ⋅Buffer B: 0.5% acetic acid in H₂O; ⋅Buffer C: 0.5% acetic acid, 60% acetonitrile and 40% H₂O; ⋅Buffer D: 0.5% acetic acid, 80% acetonitrile and 20% H₂O.
Adaptor (MiniSpin Column Collar, come with MicroSpin columns; The Nest Group, Inc., MA; cat. no. SUM SS18V).
Empore C18 Extraction disks (3M, MN; cat. No. 2215).

### Protocol Steps:

1. Single or multiple layers of C18 Extraction disks are packed into the tips, as necessary.
2) Packed tips are placed with the adaptor into the 2.0 mL microtubes (as shown in FIG. 4).
3) Conditioning I: load 200 μL buffer A (methanol) into the tips; spin at 4000 rpm for ˜1 min;
Conditioning II: load 200 μL buffer D (0.5% acetic acid, 80% acetonitrile and 20% H₂O) into the tips, spin at 4000 rpm for ˜1 min.
4) Equilibration: load 200 μL buffer B (0.5% acetic acid in H₂O) into the tips, spin at 4000 rpm for ˜1 min.
5) Resuspend the dried peptide samples into 100 μL of buffer B, and vortex for around 10 min. The peptides may come from in-gel digestion, in-solution digestion, filter aided sample preparation (FASP) or 96FASP 16.
6) Binding: load 100 μL solution in the tips and spin at 4000 rpm for about 1.5 min. Re-load the flow-through into the tips and spin again. Repeat this binding step 2-3 times.
7) Wash: load 200 μL buffer B and spin at 4000 rpm for 2-3 min. Discard the flow-through.
8) Elution: place the StageTips into new collection tubes; load 200 μL buffer C, spin at 4000 rpm for ˜2 min; load 200 μL buffer D, spin at 4000 rpm for ˜2 min, repeat elution with buffer D one more time. The total volume of the elution is ˜600 μL.
9) Dry the peptide elutes in a Speed-Vac, re-suspend with HPLC buffer for immediate analysis, or store at ˜80° C. until further use.

In yet other embodiments, the described fraction is extracellular matrix (ECM) from placental ASC cultured in a bioreactor. In yet other embodiments, the described fraction is a fraction of ECM from placental ASC cultured in a bioreactor. Those skilled in the art will appreciate that ECM refers to a matrix of proteins and other molecules secreted by cells.

Compositions Comprising Exosomes

In still another embodiment, there is a provided a method for a herein-described indication, utilizing a composition comprising exosomes or other extracellular vesicles derived from a cultured placental ASC. In certain embodiments, the composition is prepared as described herein.

Micro-Needling Methods and Compositions

In other embodiments, there is a provided a cosmetic or aesthetic treatment, e.g. for a herein-described indication, comprising micro-needling the skin of a subject and subsequently applying a herein-described composition. The composition may be, in various embodiments, a lotion, a foam, a gel, a solution, or a suspension, or, in still other embodiments, any other composition described herein. In certain embodiments, the composition comprises placental ASC, or in other embodiments, lysate thereof, ASC-CM, or a fraction derived therefrom. Alternatively or in addition, the treatment is for repairing aging skin, repairing dry skin, restoring a compromised skin barrier, or stimulating hair growth.

In other embodiments, there is a provided a cosmetic or aesthetic treatment kit, e.g. for a herein-described indication, comprising a micro-needling apparatus and a herein-described composition. The composition may be, in various embodiments, a lotion, a foam, a gel, a solution, or a suspension, or, in still other embodiments, any other composition described herein. In certain embodiments, the composition comprises placental ASC, or in other embodiments, lysate thereof, ASC-CM, or a fraction thereof. Alternatively or in addition, the treatment is for repairing aging skin, repairing dry skin, restoring a compromised skin barrier, or stimulating hair growth.

Micro-needling apparatuses are known in the art, and are available, for example, from Dermaroller® (Vancouver, Canada).

Methods of Expanding ASC

Those skilled in the art will appreciate that growth media are utilized to expand the placental ASC described herein and/or produce the described CM for the compositions and methods described herein. Non-limiting examples of base media useful in 2D and 3D culturing include Minimum Essential Medium Eagle, ADC-1, LPM (Bovine Serum Albumin-free), F10(HAM), F12 (HAM), DCCM1, DCCM2, RPMI 1640, BGJ Medium (with and without Fitton-Jackson Modification), Basal Medium Eagle (BME—with the addition of Earle's salt base), Dulbecco's Modified Eagle Medium (DMEM-without serum), Yamane, IMEM-20, Glasgow Modification Eagle Medium (GMEM), Leibovitz L-15 Medium, McCoy's 5A Medium, Medium M199 (M199E—with Earle's sale base), Medium M199 (M199H—with Hank's salt base), Minimum Essential Medium Eagle (MEM-E—with Earle's salt base), Minimum Essential Medium Eagle (MEM-H—with Hank's salt base) and Minimum Essential Medium Eagle (MEM-NAA with non-essential amino acids), among numerous others, including medium 199, CMRL 1415, CMRL 1969, CMRL 1066, NCTC 135, MB 75261, MAB 8713, DM 145, Williams' G, Neuman & Tytell, Higuchi, MCDB 301, MCDB 202, MCDB 501, MCDB 401, MCDB 411, MDBC 153, and mixtures thereof in any proportions. In certain embodiments, DMEM is used. These and other useful media are available from GIBCO, Grand Island, N.Y., USA and Biological Industries, Bet HaEmek, Israel, among others.

In some embodiments, the medium may be supplemented with additional substances. Non-limiting examples of such substances are serum, which is, in some embodiments, fetal serum of cows or other species, which is, in some embodiments, 5-15% of the medium volume. In certain embodiments, the medium contains 1-5%, 2-5%, 3-5%, 1-10%, 2-10%, 3-10%, 4-15%, 5-14%, 6-14%, 6-13%, 7-13%, 8-12%, 8-13%, 9-12%, 9-11%, or 9.5%-10.5% serum, which may be FBS, or in other embodiments another animal serum.

Alternatively or in addition, the medium may be supplemented by growth factors, vitamins (e.g. ascorbic acid), cytokines, salts (e.g. B-glycerophosphate), steroids (e.g. dexamethasone) and hormones e.g., growth hormone, erythropoietin, thrombopoietin, interleukin 3, interleukin 7, macrophage colony stimulating factor, c-kit ligand/stem cell factor, osteoprotegerin ligand, insulin, insulin-like growth factor, epidermal growth factor, fibroblast growth factor, nerve growth factor, ciliary neurotrophic factor, platelet-derived growth factor, and bone morphogenetic protein.

It will be appreciated that additional components may be added to the culture medium. Such components may be antibiotics, antimycotics, albumin, amino acids, and other components known to the art for the culture of cells.

The various media described herein, i.e. the 2D growth medium and the 3D growth medium, may be independently selected from each of the described embodiments relating to medium composition. In various embodiments, any medium suitable for growth of cells in a standard tissue apparatus and/or a bioreactor may be used.

It will also be appreciated that in certain embodiments, when the described ASC are intended for administration to a human subject, the cells and the culture medium (e.g., with the above-described medium additives) are substantially xeno-free, i.e., devoid of any animal contaminants e.g., mycoplasma. For example, the culture medium can be supplemented with a serum-replacement, human serum and/or synthetic or recombinantly produced factors.

ASC and Sources Thereof

In certain embodiments, the described ASC (used either per se or to produce products used in the described methods and compositions) are placenta-derived. Except where indicated otherwise, the terms “placenta”, “placental tissue”, and the like, as used herein, refer to any portion of the placenta. Placenta-derived ASC may be obtained, in various embodiments, from either fetal or, in other embodiments, maternal regions of the placenta, or in other embodiments, from both regions. More specific embodiments of maternal sources are the decidua basalis and the decidua parietalis. More specific embodiments of fetal sources are the amnion, the chorion, and the villi.

In certain embodiments, tissue specimens are washed in a physiological buffer, non-limiting examples of which are phosphate-buffered saline (PBS) and Hank's buffer. In certain embodiments, the placental tissue from which ASC are harvested includes at least one of the chorionic and decidua regions of the placenta, or, in still other embodiments, both the chorionic and decidua regions of the placenta. More specific embodiments of chorionic regions are chorionic mesenchymal and chorionic trophoblastic tissue. More specific embodiments of decidua are decidua basalis, decidua capsularis, and decidua parietalis. In a non-limiting embodiment, a mixture of maternal and fetal placental cells can be obtained by mincing whole placenta or in other embodiments a portion thereof; or, in still other embodiments, whole placenta, apart from the amnion, chorion, and/or umbilical cord.

Placental cells may be obtained, in various embodiments, from a full-term or pre-term placenta. In some embodiments, the placental tissue is optionally minced, followed by enzymatic digestion. Single-cell suspensions can be made, in other embodiments, by treating the tissue with a digestive enzyme (see below) or/and physical disruption, a non-limiting example of which is mincing and flushing the tissue parts through a nylon filter or by gentle pipetting (e.g. Falcon, Becton, Dickinson, San Jose, Calif.) with washing medium. In some embodiments, the tissue treatment includes use of a DNAse, a non-limiting example of which is Benzonase from Merck.

Optionally, residual blood is removed from the placenta before cell harvest. This may be done by a variety of methods known to those skilled in the art, for example by perfusion. The term “perfuse” or “perfusion” as used herein refers to the act of pouring or passaging a fluid over or through an organ or tissue. In certain embodiments, the placental tissue may be from any mammal, while in other embodiments, the placental tissue is human. A convenient source of placental tissue is a post-partum placenta (e.g., less than 10 hours after birth), however, a variety of sources of placental tissue or cells may be contemplated by the skilled person. In other embodiments, the placenta is used within 8 hours, within 6 hours, within 5 hours, within 4 hours, within 3 hours, within 2 hours, or within 1 hour of birth. In certain embodiments, the placenta is kept chilled prior to harvest of the cells. In other embodiments, prepartum placental tissue is used. Such tissue may be obtained, for example, from a chorionic villus sampling or by other methods known in the art. Once placental cells are obtained, they are, in certain embodiments, allowed to adhere to an adherent material (e.g., configured as a surface) to thereby isolate adherent cells. In some embodiments, the donor is 35 years old or younger, while in other embodiments, the donor may be any woman of childbearing age.

Placenta-derived cells can be propagated, in some embodiments, by using a combination of 2D and 3D culturing conditions. Conditions for propagating adherent cells in 2D and 3D culture are further described hereinbelow and in the Examples section which follows.

Those skilled in the art will appreciate in light of the present disclosure that cells may be, in some embodiments, extracted from a placenta, for example using physical and/or enzymatic tissue disruption, followed by marker-based cell sorting, and then may be subjected to the culturing methods described herein.

Treatment of Cells with Pro-Inflammatory Cytokines

In certain embodiments of the described methods and compositions, the composition of the medium is not varied during the course of the culturing process used to expand the placental ASC that are used in the described methods and compositions and/or for producing the described CM, fractions, or lysates thereof. In other words, no attempt is made to intentionally vary the medium composition by adding or removing factors or adding fresh medium with a different composition than the previous medium. Reference to varying the composition of the medium does not include variations in medium composition that automatically occur as a result of prolonged culturing, for example due to the absorption of nutrients and the secretion of metabolites by the cells therein, as will be appreciated by those skilled in the art.

In other embodiments, the method used to expand the steps comprises 2D culturing, followed by 3D culturing. In certain embodiments, the 3D culturing method comprises the sub-steps of: (a) incubating ASC in a 3D culture apparatus in a first growth medium, wherein no inflammatory cytokines have been added to the first growth medium; and (b) subsequently incubating the ASC in a 3D culture apparatus in a second growth medium, wherein one or more pro-inflammatory cytokines have been added to the second growth medium. Those skilled in the art will appreciate, in light of the present disclosure, that the same 3D culture apparatus may be used for the incubations in the first and second growth medium by simply adding cytokines to the medium in the culture apparatus, or, in other embodiments, by removing the medium from the culture apparatus and replacing it with medium that contains cytokines. In other embodiments, a different 3D culture apparatus may be used for the incubation in the presence of cytokines, for example by moving (e.g. passaging) the cells to a different incubator, before adding the cytokine-containing medium.

Other embodiments of pro-inflammatory cytokines, and methods comprising same, are described in WO 2017/141181 to Pluristem Ltd, by Zami Aberman et al., which is incorporated by reference herein.

In still other embodiments, the described cells (which hereinafter refers to the cells used in the described methods and compositions, or, in other embodiments, the used to produce CM, or fractions thereof, that are used in the described methods and compositions) are a mixture of fetal-derived placental ASC (also referred to herein as “fetal ASC” or “fetal cells”) and maternal-derived placental ASC (also referred to herein as “maternal ASC” or “maternal cells”) and contains predominantly maternal cells. In more specific embodiments, the mixture contains at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, at least 99.92%, at least 99.95%, at least 99.96%, at least 99.97%, at least 99.98%, or at least 99.99% maternal cells, or contains between 90-99%, 91-99%, 92-99%, 93-99%, 94-99%, 95-99%, 96-99%, 97-99%, 98-99%, 90-99.5%, 91-99.5%, 92-99.5%, 93-99.5%, 94-99.5%, 95-99.5%, 96-99.5%, 97-99.5%, 98-99.5%, 90-99.9%, 91-99.9%, 92-99.9%, 93-99.9%, 94-99.9%, 95-99.9%, 96-99.9%, 97-99.9%, 98-99.9%, 99-99.9%, 99.2-99.9%, 99.5-99.9%, 99.6-99.9%, 99.7-99.9%, or 99.8-99.9% maternal cells.

In yet other embodiments, the described cells are predominantly or completely maternal cell preparations, or are predominantly or completely fetal cell preparations, each of which represents a separate embodiment. Predominantly or completely maternal cell preparations may be obtained by methods known to those skilled in the art, including the protocol detailed in Example 1 and the protocols detailed in PCT Publ. Nos. WO 2007/108003, WO 2009/037690, WO 2009/144720, WO 2010/026575, WO 2011/064669, and WO 2011/132087. The contents of each of these publications are incorporated herein by reference. Predominantly or completely fetal cell preparations may be obtained by methods known to those skilled in the art, including selecting fetal cells via their markers (e.g. a Y chromosome in the case of a male fetus), and expanding the cells. In certain embodiments, maternal cell populations are used in the described methods and compositions. In other embodiments, fetal cells are used.

In other embodiments, the described cells are a population that does not contain a detectable amount of maternal cells and is thus entirely fetal cells. A detectable amount refers to an amount of cells detectable by FACS, using markers or combinations of markers present on maternal cells but not fetal cells, as described herein. In certain embodiments, “a detectable amount” may refer to at least 0.1%, at least 0.2%, at least 0.3%, at least 0.4%, at least 0.5%, at least 0.6%, at least 0.7%, at least 0.8%, at least 0.9%, or at least 1%.

In still other embodiments, the preparation is a mixture of fetal and maternal cells and is enriched for fetal cells. In more specific embodiments, the mixture contains at least 70% fetal cells. In more specific embodiments, at least about 30%, 35%, 40%, 45%, 50%, 55%, 60%, 65%, 75%, 80%, 85%, 90%, 95%, 96%, 97%, 98%, 99% or 100% of the cells are fetal cells. Expression of CD200, as measured by flow cytometry, using an isotype control to define negative expression, can be used as a marker of fetal cells under some conditions. In yet other embodiments, at least 60%, at least 65%, at least 70%, at least 75%, at least 80%, at least 85%, at least 90%, at least 92%, at least 95%, at least 96%, at least 97%, at least 98%, at least 99%, at least 99.5%, at least 99.7%, or at least 99.9% of the described cells are fetal cells.

In more specific embodiments, the mixture contains 20-80% fetal cells; 30-80% fetal cells; 40-80% fetal cells; 50-80% fetal cells; 60-80% fetal cells; 20-90% fetal cells; 30-90% fetal cells; 40-90% fetal cells; 50-90% fetal cells; 60-90% fetal cells; 20-80% maternal cells; 30-80% maternal cells; 40-80% maternal cells; 50-80% maternal cells; 60-80% maternal cells; 20-90% maternal cells; 30-90% maternal cells; 40-90% maternal cells; 50-90% maternal cells; or 60-90% maternal cells.

In certain embodiments, the described ASC are distinguishable from mesenchymal stromal cells (MSC), which may, in some embodiments, be isolated from bone marrow. In further embodiments, the cells are human MSC as defined by The Mesenchymal and Tissue Stem Cell Committee of the International Society for Cellular Therapy (Dominici et al., 2006), based on the following 3 criteria: 1. Plastic-adherence when maintained in standard culture conditions (a minimal essential medium+20% fetal bovine serum (FBS)). 2. Expression of the surface molecules CD105, CD73 and CD90, and lack of expression of CD45, CD34, CD14 or CD1 lb, CD79α or CD19 and HLA-DR. 3. Ability to differentiate into osteoblasts, adipocytes and chondroblasts in vitro. By contrast, the described placental cells are, in certain embodiments, characterized by a reduced differentiation potential, as exemplified and described further herein.

Serum-Free and Serum Replacement Media

In other embodiments, the described cell populations are produced by expanding a population of placental ASC in a medium that contains less than 5% animal serum. In certain embodiments, the cell population contains at least predominantly fetal cells (referred to as a “fetal cell population”), or, in other embodiments, contains at least predominantly maternal cells (a “maternal cell population”). In other embodiments, factors obtained from the maternal, or in other embodiments fetal, cells are used in the described methods and compositions.

In certain embodiments, the aforementioned medium contains less than 4%; less than 3%; less than 2%; less than 1%; less than 0.5%; less than 0.3%; less than 0.2%; or less than 0.1% animal serum. In other embodiments, the medium does not contain animal serum. In other embodiments, the medium is a defined medium to which no serum has been added. Low-serum and serum-free media are collectively referred to as “serum-deficient medium/media”.

Those skilled in the art will appreciate that reference herein to animal serum includes serum from a variety of species, provided that the serum stimulates expansion of the ASC population. In certain embodiments, the serum is mammalian serum, non-limiting examples of which are human serum, bovine serum (e.g. fetal bovine serum and calf bovine serum), equine serum, goat serum, and porcine serum.

In other embodiments, the described cell populations are produced by a process comprising: a. incubating the ASC population in a first medium, wherein the first medium contains less than 5% animal serum, thereby obtaining a first expanded cell population; and b. incubating the first expanded cell population in a second medium, wherein the second medium also contains less than 5% animal serum, and wherein one or more activating components are added to the second medium. This second medium can also be referred to herein as an activating medium. In other embodiments, the first medium or the second medium, or in other embodiments both the first and second medium, is/are serum free. In still other embodiments, the first medium contains a first basal medium, with the addition of one or more growth factors, collective referred to as the “first expansion medium” (to which a small concentration of animal serum is optionally added); and the activating medium contains a second basal medium with the addition of one or more growth factors (the “second expansion medium”), to which activating component(s) are added. In more specific embodiments, the second expansion medium is identical to the first expansion medium; while in other embodiments, the second expansion medium differs from the first expansion medium in one or more components.

In certain embodiments, the aforementioned step of incubating the ASC population in a first medium is performed for at least 17 doublings, or in other embodiments at least 6, 8, 12, 15, or at least 18 doublings; or 12-30, 12-25, 15-30, 15-25, 16-25, 17-25, or 18-25 doublings.

In other embodiments, the ASC population is incubated in the aforementioned first medium for a defined number of passages, for example 2-3, or in other embodiments 1-4, 1-3, 1-2, or 2-4; or a defined number of population doublings, for example 4-7, or in other embodiments at least 4, at least 5, at least 6, at least 7, at least 8, 4-10, 4-9, 4-8, 5-10, 5-9, or 5-8. The cells are then cryopreserved, then subjected to additional culturing in the first medium. In some embodiments, the additional culturing in the first medium is performed for 6-10 population doublings, or in other embodiments at least 6, at least 7, at least 8, at least 9, at least 10, 6-20, 7-20, 8-20, 9-20, 10-20, 6-15, 7-15, 8-15, 9-15, or 10-15 population doublings. Alternatively, the additional culturing in the first medium is performed for 2-3 passages, or in other embodiments at least 1, at least 2, at least 3, 1-5, 1-4, 1-3, 2-5, or 2-4 passages.

In still other embodiments, the step of incubating the first expanded cell population in a second medium is performed for a defined number of total passages, for example 3-5 passages, or in other embodiments 1-4, 1-3, 2-3, 2-5, or 2-4; or a defined number of total population doublings, for example 12-20, or in other embodiments 12-15, or in other embodiments 15-20, 12-18, 12-16, 14-20, or 14-18 doublings.

In other embodiments, the ASC population is incubated in the second medium for a defined number of days, for example 4-10, 5-10, 6-10, 4-9, 4-8, 4-7, 5-9, 5-8, 5-7, 6-10, 6-9, or 6-8; or a defined number of population doublings, for example at least 3, at least 4, at least 5, at least 6, 3-10, 3-9, 3-8, 4-10, 4-9, or 4-8. The cells are then subjected to additional culturing in the second medium in a bioreactor. In some embodiments, the bioreactor culturing is performed for at least 4, at least 5, at least 6, at least 7, at least 8, at least 9, 4-10, 4-9, 4-8, 5-10, 5-9, 5-8, 6-10, 6-9, or 6-8 population doublings; or, in other embodiments, for at least 4, at least 5, at least 6, at least 7, 4-15, 4-12, 4-10, 4-9, 4-8, 4-7, 4-15, 5-12, 5-10, 5-9, 5-8, 5-7, 6-15, 6-12, 6-10, 6-9, 6-8, or 6-7 days. In certain embodiments, the bioreactor contains 3D carriers, on which the cells are cultured.

In certain embodiments, the aforementioned two-stage incubation is preceded by culturing in a medium containing over 5% animal serum (e.g. as described herein). In general, for such embodiments, the nomenclature of the aforementioned steps is retained. Thus, the first medium (containing less than 5% animal serum) still retains its designation as the “first medium”, and the activating medium retains its designation as the “second [or activating] medium”.

In certain embodiments, the described serum-deficient medium is supplemented with factors intended to stimulate cell expansion in the absence of serum. Such medium is referred to herein as serum-replacement medium or SRM, and its use, for example in cell culture and expansion, is known in the art, and is described, for example, in Kinzebach et al.

SRM formulations include MSC Nutristem® XF full medium (including the supplement) and MSC Nutristem® XF basal medium (Biological Industries); Stempro® SFM and Stempro® SFM-XF (Thermo Fisher Scientific); PPRF-msc6; D-hESF10; TheraPEAK™ MSCGM-CD™ (Lonza, cat. no. 190632); and MesenCult-XF (Stem Cell Technologies, cat. no. 5429). The StemPro® media contain PDGF-BB, bFGF, and TGF-β, and insulin (Chase et al.). The composition of PPRF-msc6 is described in US 2010/0015710, which is incorporated herein by reference. D-hESF10 contains insulin (10 mcg/ml); transferrin (5 mcg/ml); oleic acid conjugated with bovine albumin (9.4 mcg/ml); FGF-2 (10 ng/ml); and TGF-β1 (5 ng/ml), as well as heparin (1 mg/ml) and standard medium components (Mimura et al.).

In still other embodiments, a chemically-defined medium is utilized. A non-limiting example of a chemically-defined medium contains DMEM/F-12 supplemented with 50 ng/ml PDGF-BB, 15 ng/ml bFGF, and 2 ng/ml TGF-β. This medium yielded similar results to Stempro® SFM-XF. DMEM/F-12 is a known basal medium, available commercially from Thermo Fisher Scientific (cat. no. 10565018).

In certain embodiments, the described SRM comprises bFGF (basic fibroblast growth factor, also referred to as FGF-2), TGF-β (TGF-β, including all isotypes, for example TGFβ1, TGFβ2, and TGFβ3), or a combination thereof. In other embodiments, the SRM comprises bFGF, TGF-β, and PDGF. In still other embodiments, the SRM comprises bFGF and TGF-β, and lacks PDGF-BB. Alternatively or in addition, insulin is also present. In still other embodiments, an additional component selected from ascorbic acid, hydrocortisone and fetuin is present; 2 components selected from ascorbic acid, hydrocortisone and fetuin are present; or ascorbic acid, hydrocortisone and fetuin are all present.

In other embodiments, the described SRM comprises bFGF, TGF-β, and insulin. In additional embodiments, a component selected from transferrin (5 micrograms/milliliter [mcg/ml]) and oleic acid are present; or both transferrin and oleic acid are present. Oleic acid can be, in some embodiments, conjugated with a protein, a non-limiting example of which is albumin. In some embodiments, the SRM comprises 5-20 ng/ml bFGF, 2-10 ng/ml TGF-β, and 5-20 ng/ml insulin, or, in other embodiments, 7-15 ng/ml bFGF, 3-8 ng/ml TGF-β, and 7-15 ng/ml insulin. In more specific embodiments, a component selected from 2-10 mcg/ml transferrin and 5-20 mcg/ml oleic acid, or in other embodiments, a component selected from 3-8 mcg/ml transferrin and 6-15 mcg/ml oleic acid, or in other embodiments the aforementioned amounts of both components (transferrin and oleic acid) is/are also present.

In still other embodiments, the SRM further comprises a component, or in other embodiments 2, 3, or 4 components, selected from ethanolamine, glutathione, ascorbic acid, and albumin. Alternatively or in addition, the SRM further comprises a trace element, or in other embodiments, 2, 3, 4, or more than 4 trace elements. In some embodiments, the trace element(s) are selected from selenite, vanadium, copper, and manganese.

In yet other embodiments, the described SRM comprises bFGF and EGF. In more specific embodiments, the bFGF and EGF are present at concentrations independently selected from 5-40, 5-30, 5-25, 6-40, 6-30, 6-25, 7-40, 7-30, 7-25, 7-20, 8-, 8-17, 8-15, 8-13, 9-20, 9-17, 9-15, 10-15, 5-20, 5-10, 7-13, 8-12, 9-11, or 10 ng/ml. In certain embodiments, insulin; and/or transferrin is also present. In more specific embodiments, the insulin and transferrin are present at respective concentrations of 5-20 and 2-10; 6-18 and 3-8; or 8-15 and 4-7 mcg/ml. Alternatively or in addition, the SRM further comprises an additional component selected from BSA, selenite (e.g. sodium selenite), pyruvate (e.g. sodium pyruvate); heparin, and linolenic acid. In other embodiments 2 or more, or in other embodiments 3 or more, in other embodiments 4 or more, or in other embodiments all 5 of BSA, selenite, pyruvate, heparin, and linolenic acid are present. In more specific embodiments, the BSA, selenite, pyruvate, heparin, and linolenic acid are present at respective concentrations of 0.1-5%, 2-30 ng/mL, 5-25 mcg/ml, 0.05-0.2 mg/ml, and 5-20 nM; or in other embodiments at respective concentrations of 0.2-2%, 4-10 ng/mL, 7-17 mcg/ml, 0.07-0.15 mg/ml, and 7-15 nM; or in other embodiments the aforementioned amounts or 2 or more, or in other embodiments 3 or more, in other embodiments 4 or more, or in other embodiments all 5 of BSA, selenite, pyruvate, heparin, and linolenic acid are present.

In other embodiments, bFGF, where present, is present at a concentration of 1-40, 1-30, 1-20, 2-40, 2-30, 2-20, 3-40, 3-30, 3-20, 3-15, 4-30, 4-20, 4-15, 5-30, 5-20, 5-15, 6-14, 7-14, 8-13, 8-12, 9-11, 9-12, about 10, or 10 nanograms per milliliter (ng/ml).

In other embodiments, EGF, where present, is present at a concentration of 1-40, 1-30, 1-20, 2-40, 2-30, 2-20, 3-40, 3-30, 3-20, 3-15, 4-30, 4-20, 4-15, 5-30, 5-20, 5-15, 6-14, 7-14, 7-25, 7-22, 8-25, 8-22, 9-21, 10-20, 8-13, 8-12, 9-11, 9-12, about 10, or 10 ng/ml.

In other embodiments, TGF-β, where present, is present at a concentration of 1-25, 2-25, 3-25, 4-25, 5-25, 1-20, 1-15, 1-10, 1-8, 1-7, 1-6, 1-5, 2-20, 2-15, 2-10, 3-20, 3-15, 3-10, 3-8, 3-7, 4-8, 4-7, 4-6, 4.5-5.5, about 5, or 5 ng/ml.

In other embodiments, PDGF, where present, is present at a concentration of 1-50, 1-40, 1-30, 1-20, 1-15, 1-10, 1-8, 1-7, 1-6, 1-5, 1-4, 1-3, 1-2, 2-50, 2-40, 2-30, 2-20, 2-15, 2-10, 2-8, 2-7, 2-6, 2-5, 2-4, 3-50, 3-40, 3-30, 3-20, 3-15, 3-10, 3-8, 3-7, 3-6, 3-5, 3-4, 4-40, 4-30, 4-20, 5-40, 5-30, 5-20, 5-15, 5-12, 5-10, 10-20, 10-18, 10-16, or 10-15, 2-20, about 2, about 3, about 5, about 10, about 15, about 20, 2, 3, 5, 10, 15, or 20 ng/mL.

In still other embodiments, ASC are extracted from placenta into serum-containing medium. A non-limiting extraction protocol is described in Example 1 of International Patent

Application WO 2016/098061, in the name of Esther Lukasiewicz Hagai et al., published on Jun. 23, 2016, which is incorporated herein by reference in its entirety. Following initial extractions, cells are, in further embodiments, expanded in SRM, in some embodiments for about 2-3 passages, or typically about 4-12 population doublings after the first passage. In yet further embodiments, the culturing is optionally followed by cell concentration, formulation, and cryopreservation, and the optional thawing and additional culturing. In certain embodiments, the initial culturing is all carried out on a 2D substrate. Those skilled in the art will appreciate that non-limiting examples of cryopreservation excipients include DMSO and serum. Other embodiments of cryopreservation media are described herein.

In certain embodiments, the aforementioned culturing steps are followed by culturing in a bioreactor, which is, in some embodiments, performed in SRM. In other embodiments, the bioreactor contains serum-containing medium. In more particular embodiments, the bioreactor culture is performed for 2-5 additional doublings, or in other embodiments up to 10 additional doublings. In certain embodiments, the bioreactor contains a 3D substrate. In other embodiments, a platelet lysate, a non-limiting example of which is human platelet lysate, is used in place of serum. In still other embodiments, a cytokine-containing medium is used in place of the serum-containing medium.

Optionally, bioreactor growth may be followed by any or all of harvest, cell concentration, washing, formulation, and/or cryopreservation.

In other embodiments, the step of incubating the ASC population in a SFM/SPM is performed in a batch culture, and at least a portion of the subsequent step is performed under perfusion. In still other embodiments, the aforementioned subsequent step is initiated in a batch culture for a duration of 2-6, or in other embodiments at least 2, at least 3, at least 4, at least 5, at least 6, 1-5, 2-5, 3-5, 1-2, 1-3, or 1-5-cell doublings, before performing additional expansion in a serum-containing medium under perfusion.

Other SFM and SRM embodiments are disclosed in international patent application publ. no. WO 2019/186471, filed on Mar. 28, 2019, in the name of Lior Raviv et al., which is incorporated herein by reference.

Surface Markers and Additional Characteristics of ASC

Alternatively or additionally, the described ASC (which are used in the described methods and compositions, or to produce CM, lysates, or fractions thereof) may express a marker or a collection of markers (e.g. surface marker) characteristic of MSC or mesenchymal-like stromal cells. In some embodiments, the ASC express some or all of the following markers: CD105 (UniProtKB Accession No. P17813), CD29 (UniProtKB Accession No. P05556), CD44 (UniProtKB Accession No. P16070), CD73 (UniProtKB Accession No. P21589), and CD90 (UniProtKB Accession No. P04216). In some embodiments, the ASC do not express some or all of the following markers: CD3 (e.g. UniProtKB Accession Nos. P09693 [gamma chain] P04234 [delta chain], P07766 [epsilon chain], and P20963 [zeta chain]), CD4 (UniProtKB Accession No. P01730), CD11b (UniProtKB Accession No. P11215), CD14 (UniProtKB Accession No. P08571), CD19 (UniProtKB Accession No. P15391), and/or CD34 (UniProtKB Accession No. P28906). In more specific embodiments, the ASC also lack expression of CD5 (UniProtKB Accession No. P06127), CD20 (UniProtKB Accession No. P11836), CD45 (UniProtKB Accession No. P08575), CD79-alpha (UniProtKB Accession No. B5QTD1), CD80 (UniProtKB Accession No. P33681), and/or HLA-DR (e.g. UniProtKB Accession Nos. P04233 [gamma chain], P01903 [alpha chain], and P01911 [beta chain]). The aforementioned, non-limiting marker expression patterns were found in certain maternal placental cell populations that were expanded on 3D substrates. All UniProtKB entries mentioned in this paragraph were accessed on Jul. 7, 2014. Those skilled in the art will appreciate that the presence of complex antigens such as CD3 and HLA-DR may be detected by antibodies recognizing any of their component parts, such as, but not limited to, those described herein.

In some embodiments, the ASC possess a marker phenotype that is distinct from bone marrow-mesenchymal stem cells (BM-MSC). In certain embodiments, the ASC are positive for expression of CD10 (which occurs, in some embodiments, in both maternal and fetal ASC); are positive for expression of CD49d (which occurs, in some embodiments, at least in maternal ASC); are positive for expression of CD54 (which occurs, in some embodiments, in both maternal and fetal ASC); are bimodal, or in other embodiments positive, for expression of CD56 (which occurs, in some embodiments, in maternal ASC); and/or are negative for expression of CD106. Except where indicated otherwise, bimodal refers to a situation where a significant percentage (e.g. at least 20%) of a population of cells express a marker of interest, and a significant percentage do not express the marker.

“Positive” expression of a marker indicates a value higher than the range of the main peak of an isotype control histogram; this term is synonymous herein with characterizing a cell as “express”/“expressing” a marker. “Negative” expression of a marker indicates a value falling within the range of the main peak of an isotype control histogram; this term is synonymous herein with characterizing a cell as “not express”/“not expressing” a marker. “High” expression of a marker, and term “highly express[es]” indicates an expression level that is more than 2 standard deviations higher than the expression peak of an isotype control histogram, or a bell-shaped curve matched to said isotype control histogram.

A cell is said to express a protein or factor if the presence of protein or factor is detectable by standard methods, an example of which is a detectable signal using fluorescence-activated cell sorting (FACS), relative to an isotype control. Reference herein to “secrete”/ “secreting”/“secretion” relates to a detectable secretion of the indicated factor, above background levels in standard assays. For example, 0.5×10⁶ fetal or maternal ASC can be suspended in 4 ml medium (DMEM+10% FBS+2 mM L-Glutamine), added to each well of a 6 well-plate, and cultured for 24 hrs in a humidified incubator (5% CO2, at 37° C.). After 24 h, DMEM is removed, and cells are cultured for an additional 24 hrs in 1 ml RPMI 1640 medium+2 mM L-Glutamine+0.5% HSA. The CM is collected from the plate, and cell debris is removed by centrifugation.

According to some embodiments, the described ASC are capable of suppressing an immune reaction in the subject. Methods of determining the immunosuppressive capability of a cell population are well known to those skilled in the art, and exemplary methods are described in Example 3 of PCT Publication No. WO 2009/144720, which is incorporated herein by reference in its entirety. For example, a mixed lymphocyte reaction (MLR) may be performed. In an exemplary, non-limiting MLR assay, irradiated cord blood (iCB) cells, for example human cells or cells from another species, are incubated with peripheral blood-derived monocytes (PBMC; for example human PBMC or PBMC from another species), in the presence or absence of a cell population to be tested. PBMC cell replication, which correlates with the intensity of the immune response, can be measured by a variety of methods known in the art, for example by ³H-thymidine uptake. Reduction of the PBMC cell replication when co-incubated with test cells indicates an immunosuppressive capability. Alternatively, a similar assay can be performed with peripheral blood (PB)-derived MNC, in place of CB cells. Alternatively or in addition, secretion of pro-inflammatory and anti-inflammatory cytokines by blood cell populations (such as CB cells or PBMC) can be measured when stimulated (for example by incubation with non-matched cells, or with a non-specific stimulant such as PHA), in the presence or absence of the ASC. In certain embodiments, for example in the case of human ASC, as provided in WO 2009/144720, when 150,000 ASC are co-incubated for 48 hours with 50,000 allogeneic PBMC, followed by a 5-hour stimulation with 1.5 mcg/ml of LPS, the amount of IL-10 secretion by the PBMC is at least 120%, at least 130%, at least 150%, at least 170%, at least 200%, or at least 300% of the amount observed with LPS stimulation in the absence of ASC.

In other embodiments, the ASC secrete a factor(s) that promotes angiogenesis. In certain embodiments, the ASC secrete a factor selected from VEGF (vascular endothelial growth factor), angiogenin, Angiopoietin 1, MCP-1, IL-8, Serpin E1, and GCP2/CXCL6. In other embodiments, the ASC secrete VEGF, Angiogenin, Angiopoietin 1, MCP-1, IL-8, and Serpin E1, which were found to be secreted by maternal cells. In still other embodiments, the ASC secrete VEGF, Angiogenin, Angiopoietin 1, MCP-1, IL-8, Serpin E1, and GCP2/CXCL6, which were found to be secreted by fetal cells.

In yet other embodiments, the ASC secrete anti-fibrotic factor(s). In certain embodiments, the ASC secrete a factor selected from Serpin E1 (Plasminogen activator inhibitor 1; Uniprot Accession No. P05121) and uPAR (Urokinase plasminogen activator surface receptor; Uniprot Accession No. Q03405). In other embodiments, the ASC secrete factors that facilitate. In still other embodiments, the ASC secrete Serpin E1 and uPAR, which were found to be secreted by maternal and fetal cells. All UniProt entries in this paragraph were accessed on Apr. 3, 2017.

In other embodiments, the ASC secrete a factor(s) that promotes extracellular matrix (ECM) remodeling. In certain embodiments, the ASC secrete a factor selected from TIMP1, TIMP2, MMP-1, MMP-2, and MMP-10. In other embodiments, the ASC secrete TIMP1, TIMP2, MMP-1, MMP-2, and MMP-10, which were found to be secreted by maternal cells. In still other embodiments, the ASC secrete TIMP1, TIMP2, MMP-1, and MMP-10, which were found to be secreted by fetal cells.

In other embodiments, the described ASC exhibit a spindle shape when cultured under 2D conditions.

According to some embodiments, the ASC express CD200, while in other embodiments, the ASC lack expression of CD200. In still other embodiments, less than 30%, 25%, 20%, 15%, 10%, 8%, 6%, 5%, 4%, 3%, or 2%, 1%, or 0.5% of the adherent cells express CD200. In yet other embodiments, greater than 70%, 75%, 80%, 85%, 90%, 92%, 94%, 95%, 96%, 97%, 98%, 99%, or 99.5% of the adherent cells express CD200.

In still other embodiments, the described ASC possess any other marker phenotype, other characteristic (e.g. secretion of factor(s), differentiation capability, resistance to differentiation, inhibition of T-cell proliferation, or stimulation of myoblast proliferation), or combination thereof that is mentioned in international patent application publ. no. WO 2019/239295, filed Jun. 10, 2019, to Zami Aberman et al, which is incorporated herein by reference.

In still other embodiments, the cells may be allogeneic, or in other embodiments, the cells may be autologous. In other embodiments, the cells may be fresh or, in other embodiments, frozen (for example, cryo-preserved).

In certain embodiments, any of the aforementioned ASC populations are used in the described methods and compositions. In other embodiments, lysates or CM obtained from the cells, or fractions thereof, are used in the described methods and compositions. Each population may be freely combined with each of the described aesthetic treatments, and each combination represents a separate embodiment. Furthermore, the cells utilized to generate CM or contained in the composition can be, in various embodiments, autologous, allogeneic, or xenogenic to the treated subject. Each type of cell may be freely combined with the therapeutic embodiments mentioned herein.

Additional Method Characteristics for Preparation of ASC and Lysates, CM, and Fractions Derived Therefrom

In some embodiments, the described placental ASC have been incubated in a 3D bioreactor. In some embodiments, the described fractions (e.g. exosomes) are isolated from the 3D bioreactor-produced CM, in which the ASC have been incubated. Each described embodiment for cell expansion may be combined with any of the described embodiments for therapeutic uses of ASC, CM, lysates, or exosomes derived therefrom.

In some embodiments, the described ASC or CM are/is harvested from a 3D bioreactor in which the ASC have been incubated. Alternatively or in addition, the cells are cryopreserved, and then are thawed, after which the cells are further expanded and/or CM, fractions, lysates, or exosomes are isolated therefrom. In other embodiments, after thawing, the cells are cultured in 2D culture, from which the ASC, CM, fractions, lysates, or exosomes are isolated.

In certain embodiments, the described ASC are, or have been, subject to a 3D incubation, as described further herein. In more specific embodiments, the ASC have been incubated in a 2D adherent-cell culture apparatus, prior to the step of 3D culturing. In some embodiments, ASC are then subjected to prior step of incubation in a 2D adherent-cell culture apparatus, followed by the described 3D culturing steps.

The terms “two-dimensional culture” and “2D culture” refer to a culture in which the cells are exposed to conditions that are compatible with cell growth and allow the cells to grow in a monolayer. An apparatus suitable for such growth is referred to as a “2D culture apparatus”. Such apparatuses will typically have flat growth surfaces (also referred to as a “two-dimensional substrate(s)” or “2D substrate(s)”), in some embodiments comprising an adherent material, which may be flat or curved. Non-limiting examples of apparatuses for 2D culture are cell culture dishes and plates. Included in this definition are multi-layer trays, such as Cell Factory™, manufactured by Nunc™, provided that each layer supports monolayer culture. It will be appreciated that even in 2D apparatuses, cells can grow over one another when allowed to become over-confluent. This does not affect the classification of the apparatus as “two-dimensional”.

The terms “three-dimensional culture” and “3D culture” refer to a culture in which the cells are exposed to conditions that are compatible with cell growth and allow the cells to grow in a 3D orientation relative to one another. The term “three-dimensional [or 3D] culture apparatus” refers to an apparatus for culturing cells under conditions that are compatible with cell growth and allow the cells to grow in a 3D orientation relative to one another. Such apparatuses will typically have a 3D growth surface (also referred to as a “three-dimensional substrate” or “3D substrate”), in some embodiments comprising an adherent material, which is present in the 3D culture apparatus, e.g. the bioreactor. Certain, non-limiting embodiments of 3D culturing conditions suitable for expansion of adherent stromal cells are described in PCT Application Publ. No. WO/2007/108003, which is fully incorporated herein by reference in its entirety.

In various embodiments, “an adherent material” refers to a material that is synthetic, or in other embodiments naturally occurring, or in other embodiments a combination thereof. In certain embodiments, the material is non-cytotoxic (or, in other embodiments, is biologically compatible). Alternatively or in addition, the material is fibrous, which may be, in more specific embodiments, a woven fibrous matrix, a non-woven fibrous matrix, or any type of fibrous matrix.

In still other embodiments, the described ASC are, or have been, subject to culturing conditions (e.g. a growth substate, incubation time, bioreactor, seeding density, or harvest density) mentioned in international patent application publ. no. WO 2019/239295, filed Jun. 10, 2019, to Zami Aberman et al, which is incorporated herein by reference.

In other embodiments, the length of 3D culturing is at least 4 days; between 4-12 days; in other embodiments between 4-11 days; in other embodiments between 4-10 days; in other embodiments between 4-9 days; in other embodiments between 5-9 days; in other embodiments between 5-8 days; in other embodiments between 6-8 days; or in other embodiments between 5-7 days. In other embodiments, the 3D culturing is performed for 5-15 cell doublings, in other embodiments 5-14 doublings, in other embodiments 5-13 doublings, in other embodiments 5-12 doublings, in other embodiments 5-11 doublings, in other embodiments 5-10 doublings, in other embodiments 6-15 cell doublings, in other embodiments 6-14 doublings, in other embodiments 6-13 doublings, or in other embodiments 6-12 doublings, in other embodiments 6-11 doublings, or in other embodiments 6-10 doublings.

In certain embodiments, 3D culturing can be performed in a 3D bioreactor. In some embodiments, the 3D bioreactor comprises a container for holding medium and a 3D attachment substrate disposed therein, and a control apparatus, for controlling pH, temperature, and oxygen levels and optionally other parameters. The terms attachment substrate and growth substrate are interchangeable.

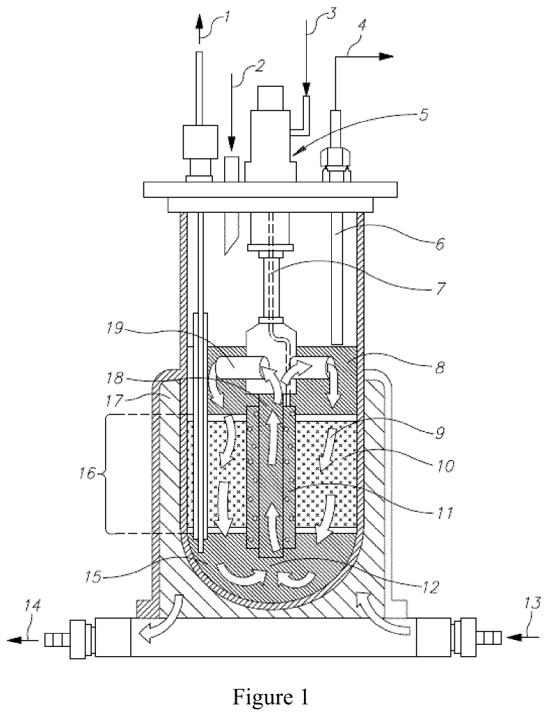

Another exemplary, non-limiting bioreactor, the Celligen 310 Bioreactor, is depicted in FIG. 1. A Fibrous-Bed Basket (16) is loaded with polyester disks (10). In some embodiments, the vessel is filled with deionized water or isotonic buffer via an external port (1 [this port may also be used, in other embodiments, for cell harvesting]) and then optionally autoclaved. In other embodiments, following sterilization, the liquid is replaced with growth medium, which saturates the disk bed as depicted in (9). In still further embodiments, temperature, pH, dissolved oxygen concentration, etc., are set prior to inoculation. In yet further embodiments, a slow stirring initial rate is used to promote cell attachment, then agitation is increased. Alternatively or addition, perfusion is initiated by adding fresh medium via an external port (2). If desired, metabolic products may be harvested from the cell-free medium above the basket (8). In some embodiments, rotation of the impeller creates negative pressure in the draft-tube (18), which pulls cell-free effluent from a reservoir (15) through the draft tube, then through an impeller port (19), thus causing medium to circulate (12) uniformly in a continuous loop. In still further embodiments, adjustment of a tube (6) controls the liquid level; an external opening (4) of this tube is used in some embodiments for harvesting. In other embodiments, a ring sparger (not visible), is located inside the impeller aeration chamber (11), for oxygenating the medium flowing through the impeller, via gases added from an external port (3), which may be kept inside a housing (5), and a sparger line (7). Alternatively or in addition, sparged gas confined to the remote chamber is absorbed by the nutrient medium, which washes over the immobilized cells. In still other embodiments, a water jacket (17) is present, with ports for moving the jacket water in (13) and out (14).

In still other embodiments, the matrix is similar to the Celligen™ Plug Flow bioreactor which is, in certain embodiments, packed with Fibra-Cel® carriers (or, in other embodiments, other carriers).

In certain embodiments, further steps of purification or enrichment for ASC may be performed. Such methods include, but are not limited to, cell sorting using markers for ASC and/or, in various embodiments, mesenchymal stromal cells or mesenchymal-like ASC.

Cell sorting, in this context, refers to any procedure, whether manual, automated, etc., that selects cells on the basis of their expression of one or more markers, their lack of expression of one or more markers, or a combination thereof. Those skilled in the art will appreciate that data from one or more markers can be used individually or in combination in the sorting process.

In more particular embodiments, cells may be removed from a 3D matrix while the matrix remains within the bioreactor. In certain embodiments, at least about 10%, 20%, or 30% of the cells are in the S and G2/M phases (collectively), at the time of harvest from the bioreactor.

In certain embodiments, the harvesting process comprises vibration or agitation, for example as described in PCT International Application Publ. No. WO 2012/140519, which is incorporated herein by reference. In certain embodiments, during harvesting, the cells are agitated at 0.7-6 Hertz, or in other embodiments 1-3 Hertz, during, or in other embodiments during and after, treatment with a protease, optionally also comprising a calcium chelator. In certain embodiments, the carriers containing the cells are agitated at 0.7-6 Hertz, or in other embodiments 1-3 Hertz, while submerged in a solution or medium comprising a protease, optionally also comprising a calcium chelator.

Those skilled in the art will appreciate that a variety of isotonic buffers may be used for washing cells and similar uses. Hank's Balanced Salt Solution (HBSS; Life Technologies) is only one of many buffers that may be used.

For any preparation used in the described methods, the therapeutically effective amount or dose can be estimated initially from in vitro and cell culture assays. Often, a dose is formulated in an animal model to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals.

The data obtained from these in vitro and cell culture assays and animal studies can be used in formulating a range of dosage for use in human. The dosage may vary depending upon the dosage form employed and the route of administration utilized. The exact formulation, route of administration and dosage can be, in some embodiments, chosen by the individual physician in view of the patient's condition.

Depending on the severity and responsiveness of the condition to be treated, dosing can be of a single or, in other embodiments, a plurality of administrations, with a course of treatment lasting from 2 days to 3 weeks or, in other embodiments, from 3 weeks to 3 months, or, in other embodiments, until alleviation of the disease state is achieved.

In certain embodiments, following administration, the majority of the cells, in other embodiments more than 60%, more than 70%, more than 80%, more than 90%, more than 95%, more than 96%, more than 97%, more than 98%, or more than 99% of the cells are no longer detectable within the subject 1 month after administration.

Formulations

In certain embodiments, the described composition is a topical composition that is manufactured by adding one or more excipients, e.g. stabilizers and penetrating-enhancing substances, to undiluted lysate, CM or a fraction thereof. In other embodiments, the described composition is a topical composition manufactured by adding one or more excipients to a concentrated lysate, CM or a fraction thereof. In other embodiments, the described composition is a topical composition manufactured by adding one or more excipients to a diluted lysate, CM or a fraction thereof. In other embodiments, the described composition is a topical composition manufactured by adding one or more excipients to a concentrated exosome preparation.

In other embodiments, the described composition is an injectable composition that is manufactured by adding 1 or more excipients, e.g. stabilizers and aqueous buffers, to placental

ASC, lysate, CM (e.g. undiluted CM) or a fraction thereof. In other embodiments, the described composition is an injectable composition manufactured by adding 1 or more excipients to a concentrated lysate, CM, or a fraction thereof. In other embodiments, the described composition is an injectable composition manufactured by adding 1 or more excipients to a diluted lysate, CM or a fraction thereof. In other embodiments, the described composition is an injectable composition manufactured by adding one or more excipients to a concentrated exosome preparation.

In other embodiments, the ASC are washed to remove serum present therewith. In more specific embodiments, the xenogenic serum components may be reduced by at least 90%, 95%, 99%, 99.5%, 99.8%, or 99.9%, or, in other embodiments, may be undetectable by standard methods, e.g. mass spectrometry.

In still other embodiments, the described lysate or CM is present at its original concentration. In other embodiments, the lysate or CM is diluted to 5-7%, 7-10%, 10-15%, 15-20%, 20-25%, 25-30%, 30-40%, 40-50%, 50-60%, 60-70%, 70-80%, 80-85%, 85-90%, 90-95%, or 95-100% of the original concentration. In other embodiments, the lysate or CM is concentrated to 150-300%, 150-400%, 150-500%, 150-200%, 120-150%, 120-300%, or 120-200% of the original concentration.

In other embodiments, the lysate, CM, or fraction is treated to remove serum present therewith. In more specific embodiments, the xenogenic serum components may be reduced by at least 90%, 95%, 99%, 99.5%, 99.8%, or 99.9%, or, in other embodiments, may be undetectable by standard methods, e.g. mass spectrometry.

In still other embodiments, the carrier of the described composition is selected from a suspension and an emulsion. In other embodiments, the carrier is selected from a cream, an ointment, a foam, a paste, a cosmetic, a cosmetic serum formulation, or an absorption base composition, each of which represents a separate embodiment.

Foams

In other embodiments, the described composition is formulated as a foam. Foam, except where indicated otherwise, refers to a dispersion in which a large proportion of gas by volume in the form of gas bubbles, is dispersed in a liquid, solid or gel. The diameter of the bubbles is usually larger than 1 micron, but the thickness of the lamellae between the bubbles is often in the usual colloidal size range, between 1 nanometer and 1 micron. In certain embodiments, a foam is used for a described lysate, CM, or fraction.

Cosmetic Serum Formulations

As will be appreciated by those skilled in the art, cosmetic serum formulations (a.k.a. cosmetic sera/serum) are topical formulations that do not contain occlusive moisturizing ingredients (such as petrolatum or mineral oil) that keep water from evaporating. They also contain fewer lubricating and thickening agents than a cream. In certain embodiments, cosmetic sera are water-based, eliminating oils altogether. In preferred embodiments, cosmetic sera exhibit rapid absorption and ability to penetrate into the deeper layers of the scalp, together with its non-greasy finish and intensive formula with a very high concentration of active substances. In certain embodiments, the cosmetic sera contains over 30%, over 40%, over 50%, over 60%, over 70%, over 80%, or over 90% active ingredient by weight. In certain embodiments, a cosmetic serum formulation is used for a described lysate, CM, or fraction.

Gels

In other embodiments, the composition is a gel. Except where indicated otherwise, gel refers to a non-fluid colloidal network or polymer network that is expanded throughout its whole volume by a fluid. In certain embodiments, ASC lysates, CM, or fractions thereof are dispersed in the gel.

Creams

In certain embodiments, the described composition is a cream. In more specific embodiments, the cream may further comprise an epidermis-penetrating agent. In other embodiments, the cream does not further comprise an epidermis-penetrating agent. In certain embodiments, the cream has a viscosity of at least 2000 centipoise, or, in other embodiments, at least 3000 centipoise, or, in other embodiments, at least 5000 centipoise, or, in other embodiments, at least 10,000 centipoise. The IUPAC definition of a cream is a highly concentrated emulsion formed by creaming of a dilute emulsion, where creaming refers to macroscopic separation of a dilute emulsion into a highly concentrated emulsion, in which interglobular contact is important, and a continuous phase under the action of gravity or a centrifugal field. This separation usually occurs upward, but the term may still be applied if the relative densities of the dispersed and continuous phases are such that the concentrated emulsion settles downward. In certain embodiments, ASC lysates, CM, or fractions thereof are dispersed in the cream.

References herein to viscosity refer to viscosity measured under standard atmospheric conditions (25° C. and pressure of 1 bar).

Epidermis-Penetrating Agents

The term epidermis-penetrating agent, except where indicated otherwise, refers to an agent that increases transport of the pharmaceutical agent or other beneficial substance into the scalp, relative to transport in the absence of the agent or substance. Non-limiting examples of penetrating agents include oleoresin capsicum or its constituents, or certain molecules containing heterocyclic rings to which are attached hydrocarbon chains. In certain embodiments, an epidermis-penetrating agent is used with a formulation comprising a described lysate, CM, or fraction.

Additional non-limiting examples of epidermis-penetrating agents include cationic, anionic, or nonionic surfactants (e.g., sodium dodecyl sulfate, polyoxamers, etc.); fatty acids and alcohols (e.g., ethanol, oleic acid, lauric acid, liposomes, etc.); anticholinergic agents (e.g., benzilonium bromide, oxyphenonium bromide); alkanones (e.g., n-heptane); amides (e.g., urea, N,N-dimethyl-m-toluamide); fatty acid esters (e.g., n-butyrate); organic acids (e.g., citric acid); polyols (e.g., ethylene glycol, glycerol); sulfoxides (e.g., dimethylsulfoxide); terpenes (e.g., cyclohexene); ureas; sugars; carbohydrates or other agents.

Still other epidermis-penetrating agents are described in U.S. Pat. No. 7,425,340, to Arnaud Grenier, Dario Norberto R Carrara, and Celine Besse; which is incorporated herein by reference.

Ointments

In other embodiments, the described active ingredients formulated in an ointment. Ointments for use in the described methods and compositions may be of a number of classes or types of ointment bases, as described, for example, in Jeannine M. Conway et al; and US Pharmacopeia. In more specific embodiments, the ointment comprises a hydrocarbon base (e.g. an oleaginous base), non-limiting examples of which are hard paraffin, soft paraffin, microcrystalline wax and ceresin. In other embodiments, the ointment comprises an absorption base, non-limiting examples of which are wool fat, beeswax, hydrophilic petrolatum, and lanolin. In other embodiments, the absorption base is a water-in-oil emulsion. In still other embodiments, the ointment comprises an oil-in-water emulsion base (e.g. a hydrophilic ointment or cream). In certain embodiments, the oil-in-water emulsion base is readily water-removable. In yet other embodiments, the ointment comprises a water-soluble base (e.g. a water-miscible base), non-limiting examples of which are macrogols 200, 300, and 400, and polyethylene glycol. In certain embodiments, the ointment lacks water-insoluble substances such as petrolatum, anhydrous lanolin, or waxes. In yet other embodiments, the ointment comprises an emulsifying base, non-limiting examples of which are emulsifying wax and cetrimide. In yet other embodiments, the ointment comprises a vegetable oil, non-limiting examples of which are olive oil, coconut oil, sesame oil, almond oil and peanut oil. In certain embodiments, the ointment has a viscosity of at least 1000 centipoise. In certain embodiments, ASC lysates, CM, or fractions thereof are dispersed in the ointment.

Lotions

In other embodiments, the described active ingredients are formulated in a lotion. Reference herein to a lotion, except where indicated otherwise, refers to a low-viscosity topical preparation intended for application to the scalp. In certain embodiments, the lotion is an water-in-oil emulsion. In other embodiments, the lotion is an oil-in-water emulsion. In certain embodiments, the lotion has a viscosity of 2000-10,000, 2000-8000, 3000-8000, 4000-7000, 5000-10,000, 5000-15,000, or 5000-20,000 centipoise. In certain embodiments, ASC lysates, CM, or fractions thereof are dispersed in the lotion.

Emulsions

In other embodiments, the described active ingredients (e.g. lysate, CM, or fraction thereof) are formulated as an emulsion. Reference herein to an emulsion, except where indicated otherwise, refers to a fluid system in which liquid droplets are dispersed within another liquid. Typically, (a) one liquid is aqueous, while the other is organic; and (b) one liquid (the dispersed phase) is dispersed in the other (the continuous phase). In some embodiments, the described emulsion is an oil/water (o/w) emulsion, wherein the dispersed phase is an organic material, and the continuous phase is water or an aqueous solution. In other embodiments, the emulsion is a water/oil (w/o) emulsion, where the dispersed phase is water or an aqueous solution, and the continuous phase is an organic liquid (an “oil”). In still other embodiments, the emulsion is a water-in-oil-in-water emulsion, or, in other embodiments, is an oil-in-water-in-oil” emulsion. Emulsions are known in the art, and are described, for example, in Khan et al, and the references cited therein. Generally, emulsions contain emulsifiers, e.g. as described herein.

In various embodiments, the droplets of the dispersed phase may be amorphous, liquid-crystalline, or a mixture thereof. Alternatively or in addition, the diameters of the droplets constituting the dispersed phase may range from 10 nm (nanometers) to 100 mcm (microns). In other embodiments, the diameters range from 10-1000 nm, or, in other embodiments, 10-700, 10-500, 10-300, 10-200, 10-150, 10-100, 10-80, 10-60, 10-50, 10-40, 20-1000, 20-700, 20-500, 20-300, 20-200, 20-150, 20-100, 20-80, 20-60, 20-50, 20-40, 30-1000, 30-700, 30-500, 30-300, 30-200, 30-150, 30-100, 30-80, 30-60, 30-50, 30-40, 50-1000, 50-700, 50-500, 50-300, 50-200, 50-150, 50-100, 50-80, 70-1000, 70-700, 70-500, 70-300, 70-200, 70-150, 70-100, 70-80, 100-1000, 100-700, 100-500, 100-300, 100-200, 100-150, 100-120, 150-1000, 150-700, 150-500, 150-300, 150-200, 200-2000, 200-1500, 200-1000, 200-700, 200-500, 200-300, 300-2000, 300-1500, 300-1000, 300-700, 300-500, 500-2000, 500-1500, 500-1000, 500-700, 700-3000, 700-2000, 700-1500, 700-1000, 1000-5000, 1000-3000, 1000-2000 nm, or 1000-1500 nm. In still other embodiments, the diameters range from 1-100 mcm, or, in other embodiments, 1-70, 1-50, 1-30, 1-20, 1-15, 1-10, 2-100, 2-70, 2-50, 2-30, 2-20, 2-15, 3-10, 3-100, 3-70, 3-50, 3-30, 3-20, 3-15, 3-10, 3-100, 3-70, 3-50, 3-30, 3-20, 3-15, 3-10, 5-100, 5-70, 5-50, 5-30, 5-20, 5-15, 5-10, 7-100, 7-70, 7-50, 7-30, 7-20, 7-15, 7-10, 10-100, 10-70, 10-50, 10-30, 10-20, 10-15, 15-100, 15-70, 15-50, 15-30, 15-20, 20-100, 20-70, 20-50, 20-30, 30-100, 30-70, 30-50, 50-100, 50-70, or 70-100 mcm.

In certain embodiments, the described emulsion is a microemulsion or nanoemulsion. Microemulsions and nanoemulsions are known in the art, and are described, for example, in Mason T G et al and the references cited therein, and in US 2019/0060185 to Thomas Doering, which is incorporated herein by reference.

Reference herein to a microemulsion, except where indicated otherwise, refers to a dispersion comprising water, oil, and a surfactant(s), that is an isotropic and thermodynamically stable system with a dispersed domain diameter from 1-100 nm, usually 10-50 nm, which can form spontaneously by self-assembly, upon simple mixing of the components and without requiring the high shear conditions. In various embodiments, the microemulsion is selected from oil dispersed in water, water dispersed in oil, and bicontinuous (interconnected). More specific embodiments of microemulsions are stabilized by surfactant and/or surfactant-cosurfactant (e.g., aliphatic alcohol) systems, which are present, in some embodiments, in sufficient quantities to confer thermodynamic stability.

In other embodiments, the described emulsion is a nanoemulsion. Reference herein to a nanoemulsion, except where indicated otherwise, refers to an emulsion whose dispersed droplets are in the 20-500 nm range, more preferably 20-200 nm, and is kinetically, but not thermodynamically, stable. Typically, nanoemulsions require application of mechanical shear force to form. In certain embodiments, the droplets are solid spheres, and their surface is amorphous and lipophilic with a negative charge. In other embodiments, the nanoemulsion is selected from: (a) an oil in water nanoemulsion, with a continuous aqueous phase, (b) a water in oil nanoemulsion, with a continuous oil phase, and (c) a bi-continuous nanoemulsion.

Emulsifiers

Those skilled in the art will appreciate that lotions, gels, emulsions, and other types of formulations described herein may require one or more emulsifiers. Reference herein to an emulsifier, except where indicated otherwise, indicates a substance that stabilizes an emulsion by increasing its kinetic stability. Typically, emulsifiers contain a polar or hydrophilic (water-soluble) portion and a non-polar (hydrophobic or lipophilic) portion. Emulsifiers tend to have preferential solubility in either water or in oil. Emulsifiers that are more soluble in water than oil generally facilitate formation of oil-in-water emulsions, while emulsifiers that are more soluble in oil generally favor water-in-oil emulsions. In certain embodiments, the described emulsifier reduces the surface tension of the emulsion to below 10 dynes/cm. Emulsifiers, and their use in facilitation formation of emulsions, are known the art, and are described e.g. in Manjit Jaiswal et al, and the references cited therein. In certain embodiments, a GRAS (generally recognized as safe) emulsifier is used. A list of GRAS substances is available from the USFDA's SCOGS (Select Committee on GRAS Substances).

In certain embodiments, the described emulsifier is a surface-active agent, or surfactant, which is, in more specific embodiments, selected from a cationic surfactant, anionic surfactant, zwitterionic surfactant, and amphoteric surfactant.

In certain embodiments, the emulsifier is a cationic surfactant. Cationic surfactant, except where indicated otherwise, refers to a substance that dissociates in aqueous solutions to form positively charged cations, which exhibit emulsifying properties. Non-limiting examples of cationic surfactants are benzalkonium salts, polyquaternium compounds, poly(vinyl pyridine), and co-N,N dimethyl ethyl methacrylate. In certain embodiments, a cationic surfactant is used in conjunction with a non-ionic emulgent.

In other embodiments, the emulsifier is an anionic surfactant. Anionic surfactant, except where indicated otherwise, refers to a substance that dissociates in aqueous solutions to form negatively charged cations, which exhibit emulsifying properties. Non-limiting examples of anionic surfactants are sodium stearate, sodium dodecyl sulfate, sodium lauryl benzene sulfonate, poly acrylic acid, anionic sulfate-based surfactants, and anionic sulfonate-based surfactants.

In other embodiments, the emulsifier is an amphoteric surfactant. Amphoteric surfactant, except where indicated otherwise, refers to a substance that possesses both positively and negatively charged groups, depending on the pH of the system. They are cationic at low pH and anionic at high pH. A non-limiting example of an amphoteric surfactant is lecithin.

In still other embodiments, the emulsifier is a non-ionic surfactant. Non-limiting examples of non-ionic surfactants include poly(ethylene oxide-b-propylene oxide), poly(ethylene oxide-b-butylene oxide), sorbitol esters of fatty acids, and ethoxylated fatty alcohol, and polysorbate-type nonionic surfactants.

In yet other embodiments, the emulsifier is a hydrophilic colloid, non-limiting examples of which are acacia, alginate, chitosan, carboxymethylcellulose, croscarmellose, microcrystalline cellulose, and xanthan gum.

In other embodiments, the emulsifier is a finely divided solid, non-limiting examples of which are bentonite and veegum.

In other embodiments, the emulsifier is a detergent, non-limiting examples of which are citric acid; dibasic ammonium citrate; calcium citrate; potassium citrate; sodium citrate; isopropyl citrate; triethyl citrate; stearyl citrate; tartaric acid, glucaric acid, mucic acid, gluconic acid, ascorbic acid, and their salts. Other embodiments include imidodiacetic acid (IDA) derivatives, e.g. nor-NTA and N-methyl dipotassium IDA.

Other embodiments of surfactants include alkyl polyglucoside (“APG”) surfactants, non-limiting examples of which are the alkylpolysaccharides that are disclosed in U.S. Pat. No. 5,776,872 to Giret et al.; U.S. Pat. No. 5,883,059 to Furman et al.; U.S. Pat. No. 5,883,062 to Addison et al.; and U.S. Pat. No. 5,906,973 to Ouzounis et al., which are all incorporated by reference. Suitable alkyl polyglucosides for use herein are also disclosed in U.S. Pat. No. 4,565,647 to Llenado describing alkylpolyglucosides having a hydrophobic group containing from about 6 to about 30 carbon atoms, or from about 10 to about 16 carbon atoms and polysaccharide, e.g., a polyglycoside, hydrophilic group containing from about 1.3 to about 10, or from about 1.3 to about 3, or from about 1.3 to about 2.7 saccharide units. Optionally, there can be a polyalkyleneoxide chain joining the hydrophobic moiety and the polysaccharide moiety. A suitable alkyleneoxide is ethylene oxide. Typical hydrophobic groups include alkyl groups, either saturated or unsaturated, branched or unbranched containing from about 8-18, or from about 10-16, carbon atoms. Suitably, the alkyl group can contain up to about 3 hydroxy groups and/or the polyalkyleneoxide chain can contain up to about 10, or less than about 5, alkyleneoxide moieties. Suitable alkyl polysaccharides are octyl, nonyldecyl, undecyldodecyl, tridecyl, tetradecyl, pentadecyl, hexadecyl, heptadecyl, and octadecyl, di-, tri-, tetra-, penta-, and hexaglucosides, galactosides, lactosides, glucoses, fructosides, fructoses and/or galactoses. Suitable mixtures include coconut alkyl, di-, tri-, tetra-, and pentaglucosides and tallow alkyl tetra-, penta-, and hexaglucosides.

Other surfactants are described in US 2008/0318822 to Maria Ochomogo et al, which is incorporated herein by reference.

Those skilled in the art will appreciate that, in some embodiments, lotions and creams can be manufactured in the following two-stage process: 1). Emollients (moisturizers) and lubricants are dispersed in oil with blending and thickening agents; and 2) Perfume, color, and preservatives (all optional) are dispersed in the water cycle. Active ingredients are broken up in both cycles depending on the raw materials involved and the desired properties of the lotion or cream.

In other embodiments, oil-in-water emulsions are manufactured by the following process: 1). Add flake/powder ingredients to the oil being used to prepare the oil phase; 2) Disperse the active ingredients; 3) Prepare the water phase containing emulsifiers and stabilizers; 4) Mix the oil and water to form an emulsion, in some cases with heating to between (45-85° C.); and 5) Continue mixing until the end product is achieved.

Suspensions and Colloids

In some embodiments, the described ASC (or, in other embodiments, particulate fractions of lysates or CM) are formulated as a suspension. Reference herein to a suspension, except where indicated otherwise, refers to a dispersion of solid particles in a liquid. Typically, a suspension is a heterogeneous mixture that contains solid particles sufficiently large for sedimentation. In more specific embodiments, the particles may be placental ASC, or, in other embodiments, may be agglomerates of material derived therefrom.

In other embodiments, the described ASC are formulated as a colloid, in which in which the suspended particles are smaller and do not settle. In more specific embodiments, the particles may be vesicles or agglomerates of material from placental ASC.

Nanoencapsulation

In yet other embodiments, the described placental ASC, lysates, CM, or fractions thereof are subject to nanoencapsulation. Techniques for nanoencapsulation are known in the art, and are described, for example, in US Patent Appl. Pub. Nos. 2015/0307649 to Khoee, Sepideh et al.; 2015/0147367 to Abbasi, Soleiman et al.; and 2019/0031937 in the name of Natura Cosmeticos S.A, which are all incorporated herein by reference; and in Nanoencapsulation Technologies for the Food and Nutraceutical Industries (Academic Press), edited by Seid Mandi Jafari. Non-limiting examples of nanoencapsulation technologies include encapsulation in polymeric materials, which may e.g. be selected from the group consisting of a mono epoxy compound, a polyvalent epoxy compound, or mixtures thereof (e.g. as described in US 2015/0307649); polymers created by coacervation of a cationic polyelectrolyte with an anionic polyelectrolyte (e.g. as described in US 2015/0147367); or polymers of cyanoacrylate type monomers (e.g. as described in US 2019/0031937). In still other embodiments, the ASC or other active ingredients are encapsulated in Nanolipidic Particles, non-limiting examples of which are described in US Patent Appl. Pub. Nos. 2017/0042826, 20150342226, and 2012/0195940, all to Michael W. Fountain, which are incorporated herein by reference.

In other embodiments, the described ASC active ingredients (e.g., lysates, CM, or fractions thereof) are formulated in nanospheres. Nanospheres are generally known to those skilled in the art, and are available, for example, from Dermazone Solutions (St. Petersburg, Fla.). In certain embodiments, the nanospheres comprise phospholipid moieties, non-limiting examples of which are Lyphazome® Nanospheres (Dermazone), which average 125-150 nanometers in diameter, and are available from Dermazone.

Additional Pharmaceutical Carriers

In certain embodiments, the described compositions comprise one or more additional pharmaceutically acceptable carriers. Herein, the term “pharmaceutically acceptable carrier” refers to a carrier or a diluent. In some embodiments, a pharmaceutically acceptable carrier does not cause significant irritation to a subject. In some embodiments, a pharmaceutically acceptable carrier does not abrogate the biological activity and properties of administered cells. Examples, without limitations, of carriers are propylene glycol, saline, emulsions, and mixtures of organic solvents with water. In some embodiments, the pharmaceutical carrier is an aqueous solution of saline.

In other embodiments, the composition further comprises at least one constituent to facilitate formulation, stability, and/or topical application of the composition. In more specific embodiments, the constituent comprises a flow regulating agent, a filler, an excipient, an alcohol, a preservative, a suspending agent, a stabilizer, a surfactant, an oil phase, an aqueous phase, a humectant, or a thickener. In other embodiments, the at least one additional constituent comprises colloidal silica, titanium dioxide, isopropyl alcohol, benzalkonium chloride, stearic acid, cetyl alcohol, isopropyl palmitate, methyparaben, propylparaben, sorbitan monostearate, sorbitol, polysorbate, milk, coconut oil, almond oil, lanolin, lecithin, or beeswax. In other embodiments, the described composition is a gel. In other embodiments, the composition is a lotion.

In still other embodiments, the composition comprises placental ASC in combination with an excipient selected from an osmoprotectant or cryoprotectant, an agent that protects cells from the damaging effect of freezing and ice formation. In certain embodiments, the cryoprotectant is a permeating compound, non-limiting examples of which are dimethyl sulfoxide (DMSO), glycerol, ethylene glycol, formamide, propanediol, poly-ethylene glycol, acetamide, propylene glycol, and adonitol; or may in other embodiments be a non-permeating compound, non-limiting examples of which are lactose, raffinose, sucrose, trehalose, and d-mannitol. In other embodiments, both a permeating cryoprotectant and a non-permeating cryoprotectant are present. In other embodiments, the excipient is a carrier protein, a non-limiting example of which is albumin. In still other embodiments, both an osmoprotectant and a carrier protein are present; in certain embodiments, the osmoprotectant and carrier protein may be the same compound. Alternatively or in addition, the composition is frozen. The cells may be any embodiment of ASC mentioned herein, each of which is considered a separate embodiment. In more specific embodiments, DMSO is present at a concentration of 2-5%; or, in other embodiments, 5-10%; or, in other embodiments, 2-10%, 3-5%, 4-6%; 5-7%, 6-8%, 7-9%, 8-10%. DMSO, in other embodiments, is present with a carrier protein, a non-limiting example of which is albumin, e.g. human serum albumin.

In other embodiments, for injection, the described ASC or other active ingredients may be formulated in aqueous solutions, e.g. in a physiologically compatible buffer, non-limiting examples of which are Hank's solution, Ringer's solution, and a physiological salt buffer.

Routes

In certain embodiments, the described methods and compositions are administered by the epidermal route, non-limiting examples of which are topical compositions. In other embodiments, the methods and compositions are administered by the intradermal route, non-limiting examples of which are injected compositions. In still other embodiments, the methods and compositions are administered sub-dermally, non-limiting examples of which are injected compositions. In yet other embodiments, the methods and compositions are administered subcutaneously, non-limiting examples of which are injected compositions.

In various embodiments, the described ASC are administered to the subject within 1 hour, within 2 hours, within 3 hours, within 4 hours, within 6 hours, within 8 hours, within 10 hours, within 12 hours, within 15 hours, within 18 hours, within 24 hours, within 30 hours, within 36 hours, within 48 hours, within 3 days, within 4 days, within 5 days, within 6 days, within 8 days, within 10 days, within 12 days, or within 20 days of a skin injury or, in other embodiments, a laser treatment. In more specific embodiments, the described compositions are administered 1-24, 2-24, 3-24, 4-24, 5-24, 6-24, 8-24, 10-24, 12-48, 1-48, 2-48, 3-48, 4-48, 5-48, 6-48, 8-48, 10-48, 12-48, 18-48, 24-48, 1-72, 2-72, 3-72, 4-72, 5-72, 6-72, 8-72, 10-72, 12-72, 18-72, 24-72, or 36-72 hours after a skin injury or, in other embodiments, a laser treatment. In still other embodiments, the described compositions are administered 3-48, 4-48, 5-48, or 6-48 hours after a skin injury or, in other embodiments, a laser treatment.

In various embodiments, when placental ASC are administered, engraftment of the described cells in the host is not required for the cells to exert the described therapeutic effects, each of which is considered a separate embodiment. In other embodiments, engraftment is required for the cells to exert the effect(s). For example, the cells may, in various embodiments, be able to exert a therapeutic effect, without themselves surviving for more than 3 days, more than 4 days, more than 5 days, more than 6 days, more than 7 days, more than 8 days, more than 9 days, more than 10 days, or more than 14 days after administration.

Compositions including the described preparations formulated in a compatible pharmaceutical carrier may also be prepared, placed in an appropriate container, and labeled for treatment of an indicated condition.

The described compositions may, if desired, be packaged in a container that is accompanied by instructions for administration.

It is clarified that each embodiment of the described ASC, lysates, CM, and fractions may be freely combined with each embodiment relating to a therapeutic method or pharmaceutical composition.

Subjects

In certain embodiments, the subject treated by the described methods and compositions is a human, with skin irritation, a laceration, a compromised skin barrier, or aged, wrinkled, or otherwise damaged skin. Alternatively or in addition, the subject has undergone a laser hair removal, a micro-needling treatment, mesotherapy, or another skin treatment. In other embodiments, the subject suffers from alopecia. In other embodiments, the subject exhibits excessive transepidermal water loss. In some embodiments, the subject is male. In other embodiments, the subject is female. In certain embodiments, the subject is an elderly subject, for example a subject over 60, over 65, over 70, over 75, over 80, 60-85, 65-85, or 70-85 years in age; is a pediatric subject, for example a subject under 18, under 15, under 12, under 10, under 8, under 6, under 5, under 4, under 3, or under 2 years, or under 18, 15, 12, 10, 8, 6, 5, 4, 3, 2, or 1 month in age; or is an adult subject, for example ages 18-60, 18-55, 18-50, 20-60, 20-55, 20-50, 20-45, 20-40, 20-35, 20-30, 25-60, 30-60, 40-60, or 50-60. In other embodiments, the subject is an animal. In some embodiments, treated animals include domesticated animals and laboratory animals, e.g., non-mammals and mammals, for example non-human primates, rodents, pigs, dogs, and cats. In certain embodiments, the subject is administered with additional therapeutic agents or cells.

Also disclosed herein are kits and articles of manufacture that are drawn to reagents that can be used in practicing the methods disclosed herein. The kits and articles of manufacture can include any reagent or combination of reagent discussed herein or that would be understood to be required or beneficial in the practice of the disclosed methods, including ASC. In another aspect, the kits and articles of manufacture comprise a label, instructions, and packaging material, for example for treating a disorder or therapeutic indication mentioned herein.

Additional objects, advantages, and novel features of the invention will become apparent to one ordinarily skilled in the art upon examination of the following examples, which are not intended to be limiting. Additionally, each of the various embodiments and aspects of the invention as delineated hereinabove and as claimed in the claims section below finds experimental support in the following examples.

### EXAMPLES

Reference is now made to the following examples, which together with the above descriptions illustrate certain embodiments in a non-limiting fashion.

### Example 1: Culturing and Production of Adherent Placental Cells

Placenta-derived cell populations containing over 90% maternal tissue-derived cells were prepared as described in Example 1 of International Patent Application WO 2016/098061, which is incorporated herein by reference in its entirety.

Osteogenesis and adipogenesis assays were performed on placental cells prepared as described in the previous paragraph and on BM adherent cells. In osteogenesis assays, over 50% of the BM cells underwent differentiation into osteocytes, while none of the placental-derived cells exhibited signs of osteogenic differentiation. In adipogenesis assays, over 50% of the BM-derived cells underwent differentiation into adipocytes. In contrast, none of the placental-derived cells exhibited morphological changes typical of adipocytes. These experiments were performed as described in Example 2 of WO 2016/098061, which is incorporated herein by reference.

### Example 2: Culture of Placental Cells in Serum-Free Medium (SFM)

Methods

The cell harvesting and expansion process consisted of 3 stages, followed by downstream processing steps: Stage 1, the intermediate cell stock (ICS) production; Stage 2, the thawing of the ICS and initial further culture steps; and Stage 3, the additional culture steps in the presence of serum. The downstream processing steps included harvest from flasks or bioreactor/s, cell concentration, washing, formulation, filling and cryopreservation. The procedure included periodic testing of the growth medium for sterility and contamination, all as described in international patent application publ. no. WO 2019/239295, which is incorporated herein by reference. Bone marrow migration assays were also performed as described in WO 2019/239295.

Results

Placental cells were extracted and expanded in serum-free (SF) medium for 3 passages. Cell characteristics of eight batches were assessed and were found to exhibit similar patterns of cell size and PDL (population doubling level since passage 1) as shown for a representative batch in Table 1. Cells also significantly enhanced hematopoiesis in a bone marrow migration (BMM) assay.

TABLE 1
Characteristics of placental cells expanded in SF medium.

| | | | Total | cell | |
|---|---|---|---|---|---|
| | | | growth | size | |
| BATCH | GROUP | Passage | (days) | (μm) | PDL |
| PD200114SFM | A | 1 | 8 | 20.3 | NA |
| | | 2 | 14 | 20.9 | 3.4 |
| | | 3 | 20 | 19.7 | 7 |
| | B | 1 | 8 | 19.5 | NA |
| | | 2 | 15 | 21.5 | 3.4 |
| | | 3 | 21 | 17 | 5.1 |

| | | | |
|---|---|---|---|
| Average P 3 | 19.1 | 17.55 | 6.12 |
| % CV P 3 | 8 | 9 | 11 |

### Example 3: Osteocyte and Adipocyte Differentiation Assays

ASC were prepared as described in Example 1. BM adherent cells were obtained as described in WO 2016/098061 to Esther Lukasiewicz Hagai and Rachel Ofir, which is incorporated herein by reference in its entirety. Osteogenesis and adipogenesis assays were performed as described in WO 2016/098061.

Osteocyte Induction.

Incubation of BM-derived adherent cells in osteogenic induction medium resulted in differentiation of over 50% of the BM cells, as demonstrated by positive alizarin red staining. On the contrary, none of the placental-derived cells exhibited signs of osteogenic differentiation.

Next, a modified osteogenic medium comprising Vitamin D and higher concentrations of dexamethasone was used. Over 50% of the BM cells underwent differentiation into osteocytes, while none of the placental-derived cells exhibited signs of osteogenic differentiation.

Adipocyte Induction.

Adipocyte differentiation of placenta- or BM-derived adherent cells in adipocyte induction medium resulted in differentiation of over 50% of the BM-derived cells, as demonstrated by positive oil red staining and by typical morphological changes (e.g. accumulation of oil droplets in the cytoplasm). In contrast, none of the placental-derived cells differentiated into adipocytes.

Next, a modified medium containing a higher indomethacin concentration was used. Over 50% of the BM-derived cells underwent differentiation into adipocytes. In contrast, none of the placental-derived cells exhibited morphological changes typical of adipocytes.

### Example 4: Further Osteocyte and Adipocyte Differentiation Assays

ASC were prepared as described in Example 2. Adipogenesis and Osteogenesis were assessed using the STEMPRO® Adipogenesis Differentiation Kit (GIBCO, Cat #A1007001) and the STEMPRO® Osteogenesis Differentiation Kit (GIBCO, Cat # A1007201), respectively.

Results

Adipogenesis and Osteogenesis of placental cells grown in SRM or in full DMEM were tested. Groups are shown in Table 2.

TABLE 2
experimental groups

| Group | Product | Batch |
|---|---|---|
| A1 | BM derived MSC | BM-122 |
| | (positive control) | |
| B1 | ASC grown in SRM | PD220914SFMS3 R001 B1.2 |
| C1 | ASC grown in SRM | R050115 R01 |
| D1 | ASC grown in SRM | R280115 R01 |
| E1 | ASC grown in full DMEM | PT041011R36 |

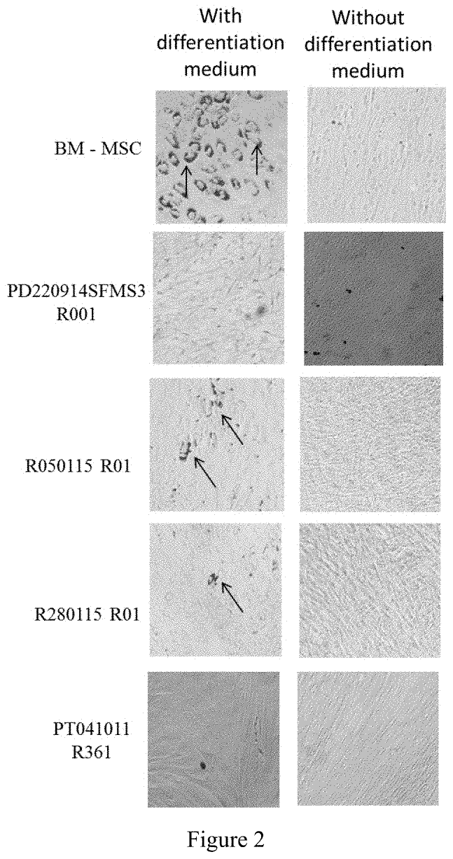

In adipogenesis assays, BM-MSCs treated with differentiation medium stained positively with Oil Red O (FIG. 2). By contrast, ⅔ of the SRM batches exhibited negligible staining, and the other SRM batch, as well as the full DMEM-grown cells, did not exhibit any staining at all, showing that they lacked significant adipogenic potential.

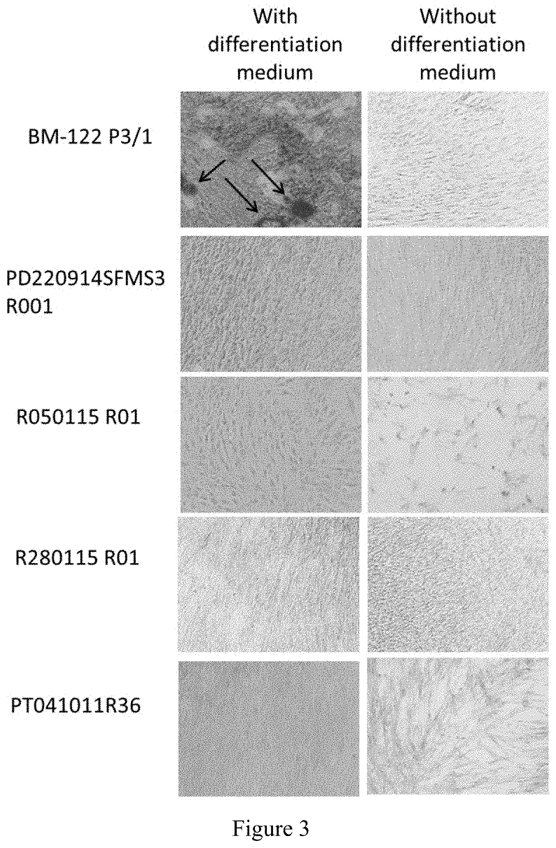

In osteogenesis assays, BM-MSCs treated with differentiation medium stained positively with Alizarin Red S (FIG. 3). By contrast, none of the placental cell batches grown in SRM or full DMEM exhibited staining, showing that the lacked significant osteogenic potential.

### Example 5: Studies of Factors Secreted by Placental ASC

CM was prepared from two batches each of maternal ASC, fetal ASC expanded in serum-containing medium, and fetal ASC expanded in SFM, after a 6-day bioreactor incubation; or a 2-day incubation in plates, changing the medium once per day.

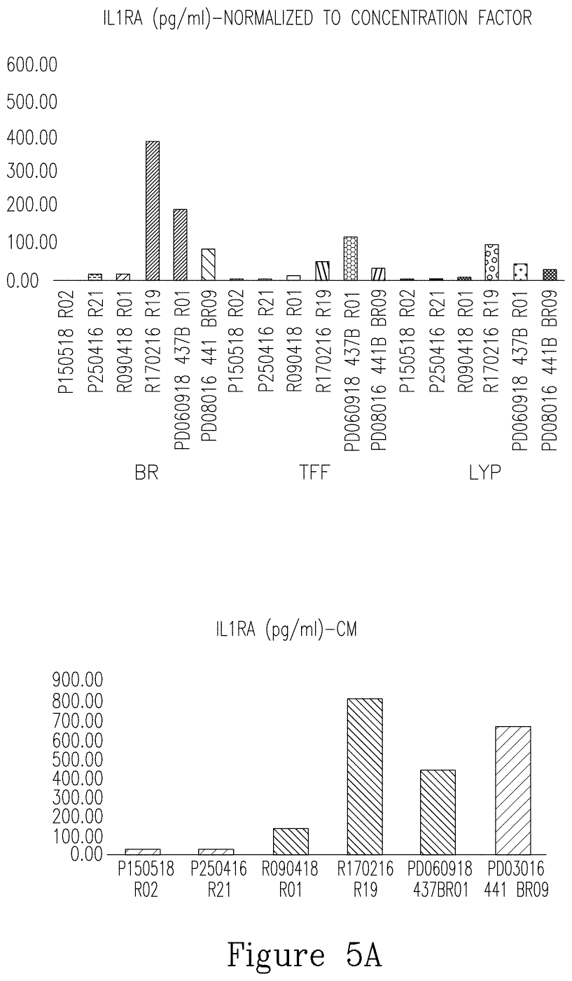

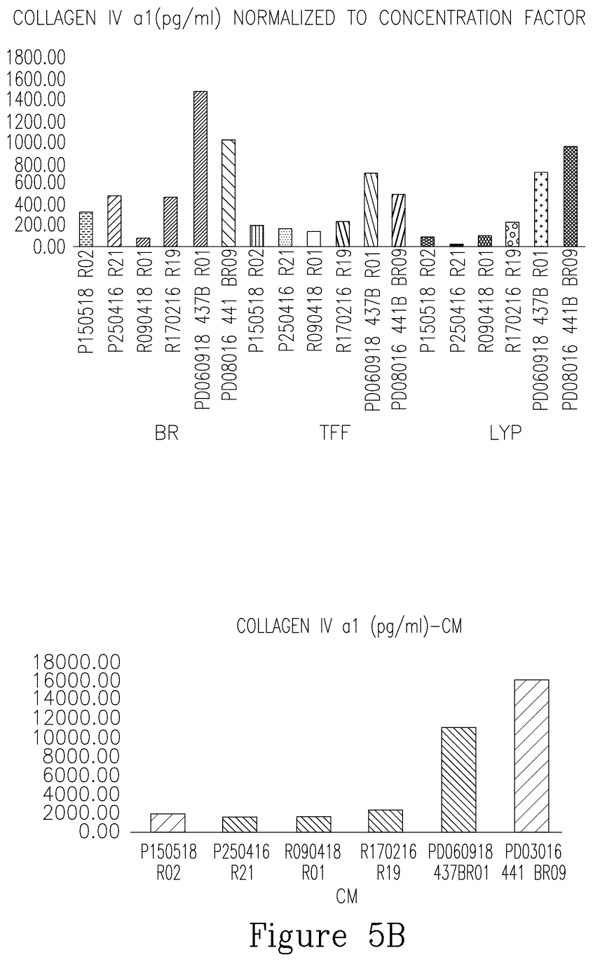

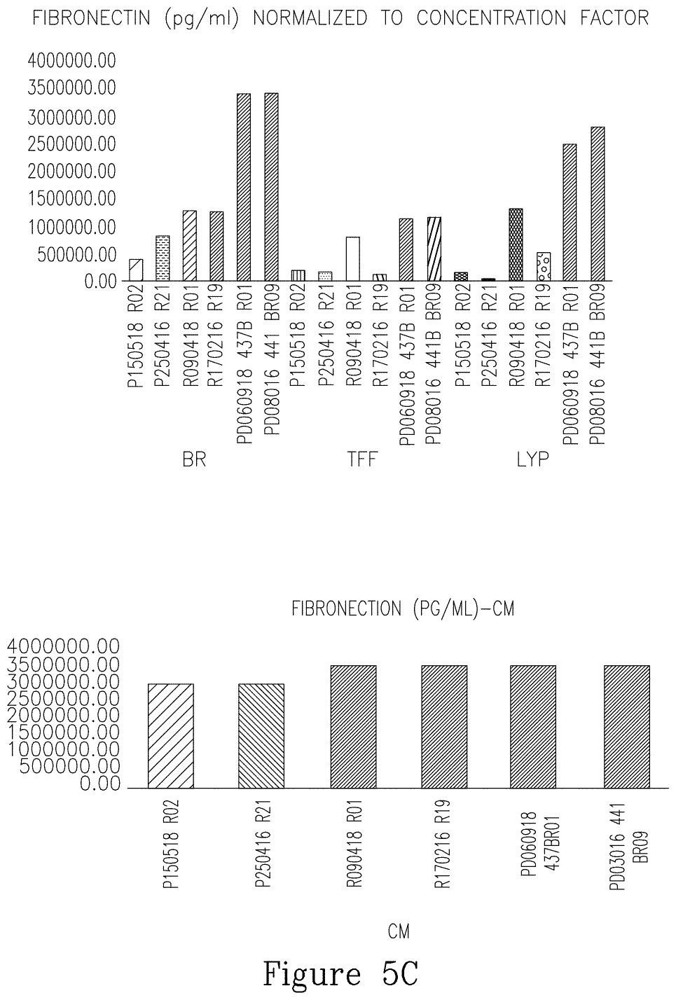

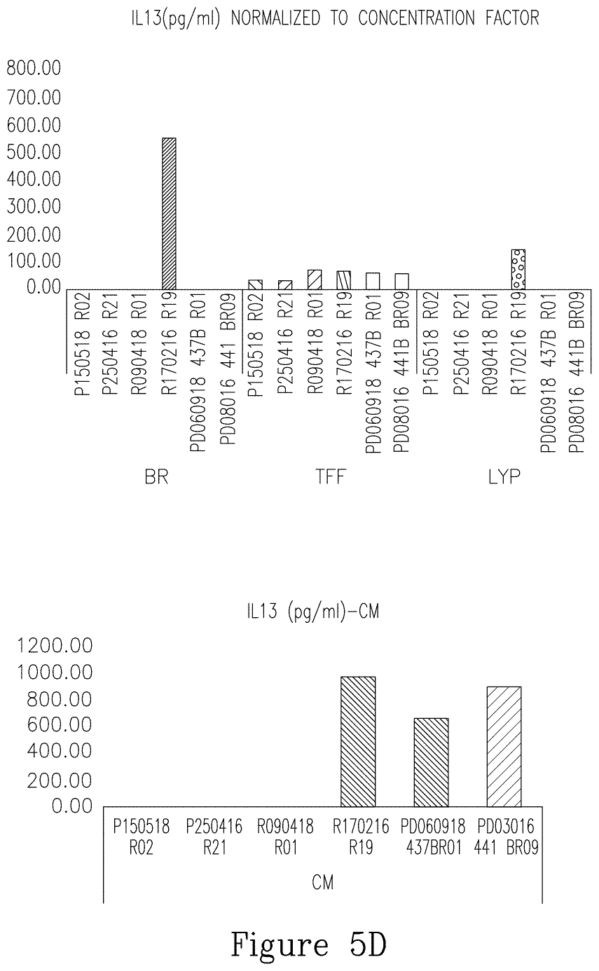

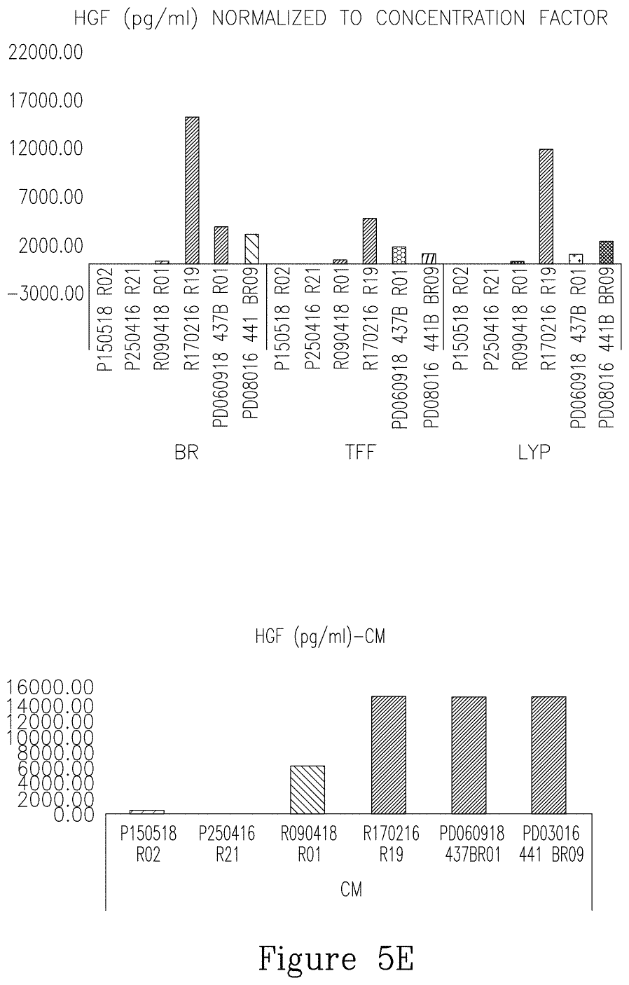

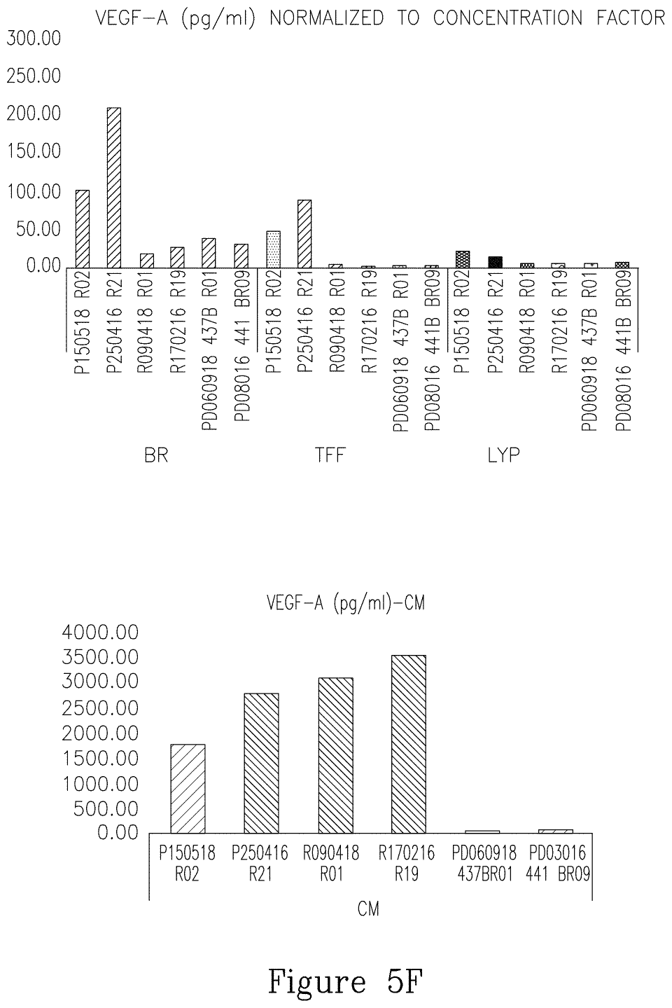

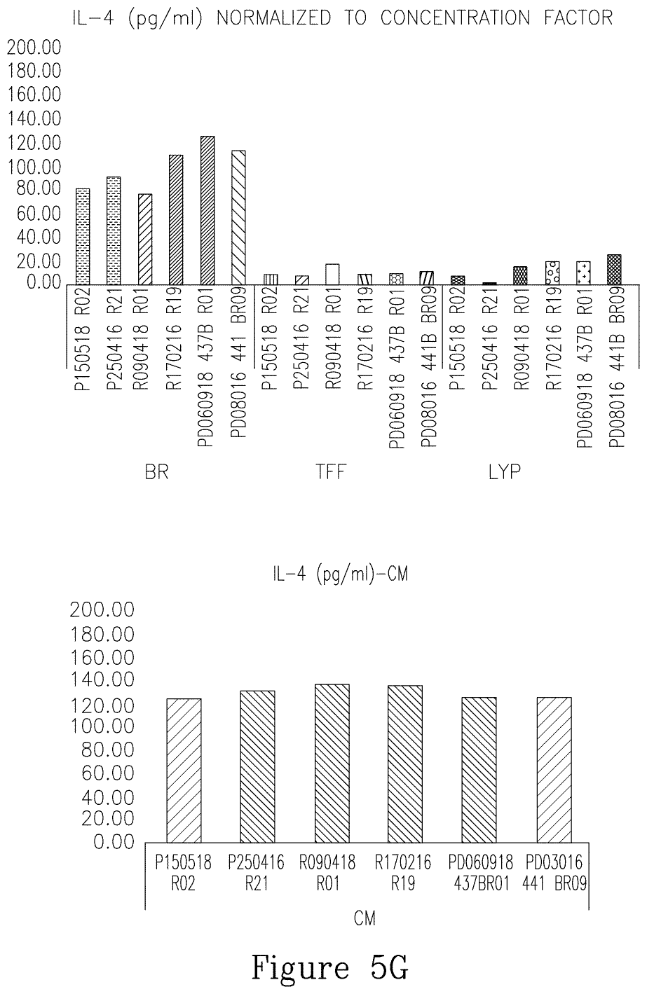

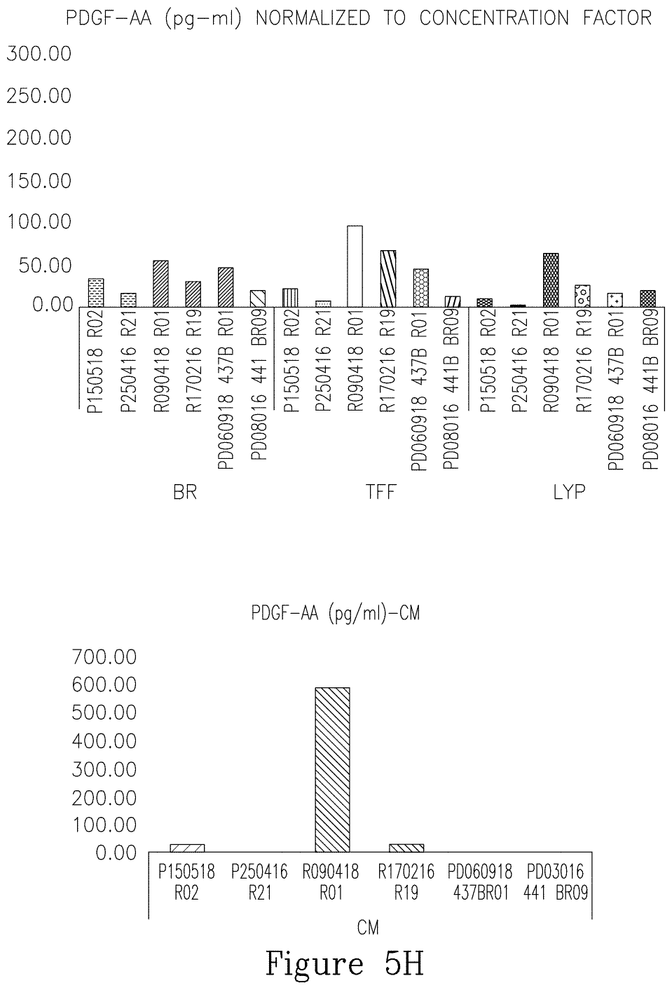

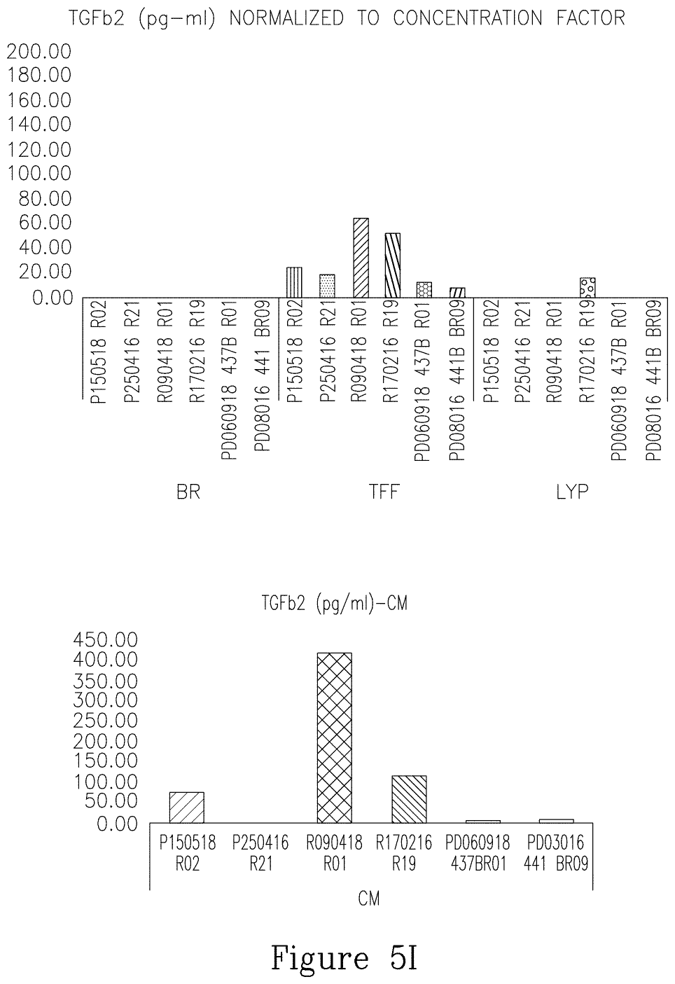

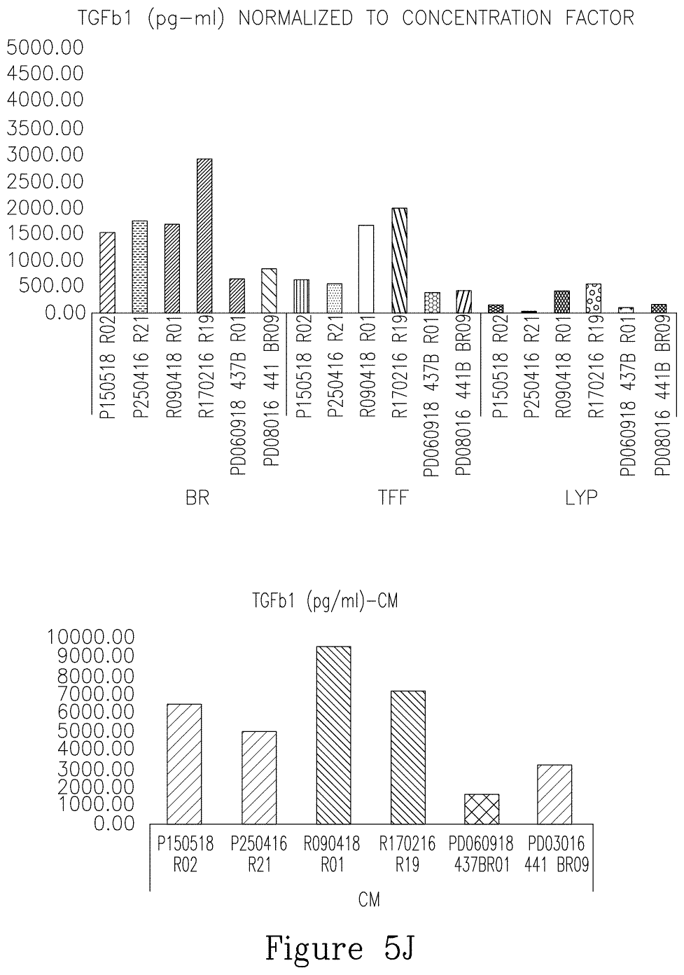

Secreted protein expression was measured by Luminex®. Collagen 1-alpha were highly expressed in all samples. IL-1-ra, Collagen IV-la, Fibronectin, IL-13, HGF, VEGF-A, IL-4, PDGF-AA, TIMP-1, TGFb2, TGFb1 were all significantly expressed in at least some samples, while IL-16 was expressed at negligible or no level (FIGS. 5A-J and Tables 3-4).

Table 3 summarizes protein expression of the indicated proteins in bioreactor media. −, +, ++, and +++ indicate <10, 10-100, 100-1000, and >1000 pg/ml, respectively.

Summary

| Protein | Maternal | Fetal/serum | Fetal/SF |
|---|---|---|---|
| Collagen 1a | +++ | +++ | +++ |
| IL-10 | − | − | − |
| EGF | − | − | − |
| IL-1RA | − | ++ | ++ |
| bFGF | − | − | ++ |
| Collagen IVa1 | ++ | ++ | +++ |
| Fibronectin | +++ | +++ | +++ |
| IL-13 | + | ++ | + |
| HGF | − | +++ | +++ |
| MMP-1 | +++ | +++ | +++ |
| MMP-2 | +++ | +++ | +++ |
| IL-16 | + | + | + |
| VEGF-A | ++ | + | + |
| IL-4 | + | + | + |
| PDGF-AA | + | + | + |
| TIMP1 | +++ | +++ | +++ |
| TGFb3 | − | − | − |
| TGFb2 | + | + | + |
| TGFb1 | +++ | +++ | +++ |

Mass spectrometry was performed on fetal/placental ASC-CM from a bioreactor incubation, and tryptic peptides of human origin were identified by their sequences. The peptides are shown in Table 4.

TABLE 4
Tryptic peptides from placental ASC-CM. “HS” refers to Homo sapiens.

| | | |
|---|---|---|
| Protein name | gene name (indicated after “GN”) | Uniprot name (in square brackets) |

Alpha-2-macroglobulin OS = HS GN = A2M PE = 1 SV = 3 − [A2MG_HUMAN]
Agrin OS = HS GN = AGRN PE = 1 SV = 5 − [AGRIN_HUMAN]
Serum albumin OS = HS GN = ALB PE = 1 SV = 1 − [A0A0C4DGB6_HUMAN]
Annexin A1 OS = HS GN = ANXA1 PE = 1 SV = 2 − [ANXA1_HUMAN]
Annexin (Fragment) OS = HS GN = ANXA2 PE = 1 SV = 1 − [H0YMM1_HUMAN]
APOC2 protein OS = HS GN = APOC2 PE = 1 SV = 1 − [Q6P163_HUMAN]
Actin-related protein 2/3 complex subunit 2 OS = HS GN = ARPC2 PE = 1 SV = 1 − [ARPC2_HUMAN]
Renin receptor (Fragment) OS = HS GN = ATP6AP2 PE = 1 SV = 1 − [A0A1BOGWD6_HUMAN]
Beta-2-microglobulin (Fragment) OS = HS GN = B2M PE = 1 SV = 1 − [H0YLF3_HUMAN]
Beta-1,4-glucuronyltransferase 1 OS = HS GN = B4GAT1 PE = 1 SV = 1 − [B4GA1_HUMAN]
Bone morphogenetic protein 1 OS = HS GN = BMP1 PE = 1 SV = 2 − [BMP1_HUMAN]
Complement C4-A OS = HS GN = C4A PE = 1 SV = 1 − [A0A0G2JPR0_HUMAN]
Calcium-binding protein 39-like OS = HS GN = CAB39L PE = 1 SV = 1 − [B7ZBJ4_HUMAN]
Cell adhesion molecule 1 OS = HS GN = CADM1 PE = 1 SV = 1 − [A0A087X0T8_HUMAN]
Capping protein (Actin filament) muscle Z-line, beta, isoform CRA_a OS = HS GN = CAPZB PE = 1 SV = 1 − [B1AK87_HUMAN]
CD44 antigen OS = HS GN = CD44 PE = 1 SV = 2 − [H0YD13_HUMAN]
Tetraspanin OS = HS GN = CD81 PE = 1 SV = 1 − [E9PJK1_HUMAN]
Cadherin-2 OS = HS GN = CDH2 PE = 1 SV = 4 − [CADH2_HUMAN]
Chymotrypsin-like elastase family member 1 OS = HS GN = CELA1 PE = 1 SV = 2 − [CELA1_HUMAN]
Collagen alpha-1(XI) chain (Fragment) OS = HS GN = COL11A1 PE = 1 SV = 8 − [C9JMN2_HUMAN]
Collagen alpha-1(XII) chain OS = HS GN = COL12A1 PE = 1 SV = 1 − [D6RGG3_HUMAN]
Collagen alpha-1(I) chain OS = HS GN = COL1A1 PE = 1 SV = 5 − [CO1A1_HUMAN]
Collagen alpha-1(III) chain OS = HS GN = COL3A1 PE = 1 SV = 4 − [CO3A1_HUMAN]
Collagen alpha-1(IV) chain OS = HS GN = COL4A1 PE = 1 SV = 3 − [CO4A1_HUMAN]
Collagen alpha-2(IV) chain OS = HS GN = COL4A2 PE = 1 SV = 4 − [CO4A2_HUMAN]
Collagen alpha-1(VI) chain OS = HS GN = COL6A1 PE = 1 SV = 1 − [A0A087X0S5_HUMAN]
Collagen alpha-3(VI) chain OS = HS GN = COL6A3 PE = 1 SV = 5 − [CO6A3_HUMAN]
Ceruloplasmin OS = HS GN = CP PE = 1 SV = 1 − [CERU_HUMAN]
Cystatin-C OS = HS GN = CST3 PE = 1 SV = 1 − [CYTC_HUMAN]
Connective tissue growth factor OS = HS GN = CTGF PE = 1 SV = 2 − [CTGF_HUMAN]
Cathepsin Z OS = HS GN = CTSZ PE = 1 SV = 1 − [CATZ_HUMAN]
Protein CutA OS = HS GN = CUTA PE = 1 SV = 1 − [C9IZG4_HUMAN]
Stromal cell-derived factor 1 OS = HS GN = CXCL12 PE = 1 SV = 1 − [SDF1_HUMAN]
Cytoplasmic FMR1-interacting protein 1 OS = HS GN = CYFIP1 PE = 1 SV = 1 − [CYFP1_HUMAN]
Protein CYR61 OS = HS GN = CYR61 PE = 1 SV = 1 − [CYR61_HUMAN]
Dermcidin OS = HS GN = DCD PE = 1 SV = 2 − [DCD_HUMAN]
Dickkopf-related protein 1 OS = HS GN = DKK1 PE = 1 SV = 1 − [DKK1_HUMAN]
Desmoglein-1 OS = HS GN = DSG1 PE = 1 SV = 2 − [DSG1_HUMAN]
Desmoplakin OS = HS GN = DSP PE = 1 SV = 3 − [DESP_HUMAN]
EF-hand domain-containing protein D2 OS = HS GN = EFHD2 PE = 1 SV = 1 − [EFHD2_HUMAN]
Eukaryotic translation initiation factor 4 gamma 1 (Fragment) OS = HS GN = EIF4G1 PE = 1 SV = 1 − [C9J6B6_HUMAN]
Eukaryotic translation initiation factor 5A OS = HS GN = EIF5A2 PE = 1 SV = 1 − [F8WCJ1_HUMAN]
Fatty acid-binding protein, heart OS = HS GN = FABP3 PE = 1 SV = 1 − [S4R3A2_HUMAN]
Fibulin-1 OS = HS GN = FBLN1 PE = 1 SV = 1 − [B1AHL2_HUMAN]
Fibrillin-1 OS = HS GN = FBN1 PE = 1 SV = 3 − [FBN1_HUMAN]
Filamin-A OS = HS GN = FLNA PE = 1 SV = 1 − [Q5HY54_HUMAN]
Fibronectin OS = HS GN = FN1 PE = 1 SV = 4 − [FINC_HUMAN]
Follistatin-related protein 1 OS = HS GN = FSTL1 PE = 1 SV = 1 − [FSTL1_HUMAN]
Rab GDP dissociation inhibitor beta OS = HS GN = GDI2 PE = 1 SV = 2 − [GDIB_HUMAN]
Glypican-1 OS = HS GN = GPC1 PE = 1 SV = 2 − [H7C410_HUMAN]
Histone H3 OS = HS GN = H3F3B PE = 1 SV = 1 − [K7EMV3_HUMAN]
HCG1745306, isoform CRA_a OS = HS GN = HBA2 PE = 1 SV = 1 − [G3V1N2_HUMAN]
Hemoglobin subunit delta OS = HS GN = HBD PE = 1 SV = 2 − [HBD_HUMAN]
Hepatocyte growth factor activator OS = HS GN = HGFAC PE = 1 SV = 1 − [HGFA_HUMAN]
Histone H2A type 1-H OS = HS GN = HIST1H2AH PE = 1 SV = 3 − [H2A1H_HUMAN]
HLA class I histocompatibility antigen, Cw-6 alpha chain OS = HS GN = HLA-C PE = 1 SV = 1 − [A0A140T9Z4_HUMAN]
Heterogeneous nuclear ribonucleoproteins A2/B1 OS = HS GN = HNRNPA2B1 PE = 1 SV = 1 − [A0A087WUI2_HUMAN]
Hornerin OS = HS GN = HRNR PE = 1 SV = 2 − [HORN_HUMAN]
Heat shock protein HSP 90-alpha OS = HS GN = HSP90AA1 PE = 1 SV = 5 − [HS90A_HUMAN]
Endoplasmin OS = HS GN = HSP90B1 PE = 1 SV = 1 − [Q96GW1_HUMAN]
Heat shock 70 kDa protein 1B OS = HS GN = HSPA1B PE = 1 SV = 1 − [HS71B_HUMAN]
Heat shock cognate 71 kDa protein OS = HS GN = HSPA8 PE = 1 SV = 1 − [E9PKE3_HUMAN]
Basement membrane-specific heparan sulfate proteoglycan core protein OS = HS GN = HSPG2 PE = 1 SV = 4 − [PGBM_HUMAN]
Serine protease HTRA1 OS = HS GN = HTRA1 PE = 1 SV = 1 − [HTRA1_HUMAN]
E3 ubiquitin-protein ligase HUWE1 OS = HS GN = HUWE1 PE = 1 SV = 3 − [HUWE1_HUMAN]
Insulin-like growth factor-binding protein 5 OS = HS GN = IGFBP5 PE = 1 SV = 1 − [IBP5_HUMAN]
Insulin-like growth factor-binding protein 6 OS = HS GN = IGFBP6 PE = 1 SV = 1 − [IBP6_HUMAN]
Insulin-like growth factor-binding protein 7 OS = HS GN = IGFBP7 PE = 1 SV = 1 − [IBP7_HUMAN]
Insulin-like growth factor I (Fragment) OS = HS GN = IGF-I PE = 1 SV = 1 − [Q13429_HUMAN]
Junction plakoglobin OS = HS GN = JUP PE = 1 SV = 3 − [PLAK_HUMAN]
Keratinocyte proline-rich protein OS = HS GN = KPRP PE = 1 SV = 1 − [KPRP_HUMAN]
Laminin subunit alpha-1 OS = HS GN = LAMA1 PE = 1 SV = 2 − [LAMA1_HUMAN]
Laminin subunit alpha-4 OS = HS GN = LAMA4 PE = 1 SV = 1 − [A0A0A0MTC7_HUMAN]
Laminin subunit beta-1 OS = HS GN = LAMB1 PE = 1 SV = 2 − [LAMB1_HUMAN]
Laminin subunit gamma-1 OS = HS GN = LAMC1 PE = 1 SV = 3 − [LAMC1_HUMAN]
Galectin-1 OS = HS GN = LGALS1 PE = 1 SV = 2 − [LEG1_HUMAN]
Galectin-3 OS = HS GN = LGALS3 PE = 1 SV = 5 − [LEG3_HUMAN]
Galectin-3-binding protein OS = HS GN = LGALS3BP PE = 1 SV = 1 − [LG3BP_HUMAN]
LIM and senescent cell antigen-like-containing domain protein 1 OS = HS GN = LIMS1 PE = 1 SV = 4 − [LIMS1_HUMAN]
Vesicular integral-membrane protein VIP36 OS = HS GN = LMAN2 PE = 1 SV = 1 − [LMAN2_HUMAN]
Protein-lysine 6-oxidase OS = HS GN = LOX PE = 1 SV = 2 − [LYOX_HUMAN]
Lysyl oxidase homolog 2 (Fragment) OS = HS GN = LOXL2 PE = 1 SV = 1 − [H0YAR1_HUMAN]
Latent-transforming growth factor beta-binding protein 2 OS = HS GN = LTBP2 PE = 1 SV = 1 − [G3V3X5_HUMAN]
Lysozyme C OS = HS GN = LYZ PE = 1 SV = 1 − [LYSC_HUMAN]
72 kDa type IV collagenase OS = HS GN = MMP2 PE = 1 SV = 2 − [MMP2_HUMAN]
Moesin OS = HS GN = MSN PE = 1 SV = 3 − [MOES_HUMAN]
Metallothionein-1E OS = HS GN = MT1E PE = 1 SV = 1 − [MT1E_HUMAN]
Matrix-remodeling-associated protein 5 OS = HS GN = MXRA5 PE = 2 SV = 3 − [MXRA5_HUMAN]
Myosin-9 OS = HS GN = MYH9 PE = 1 SV = 4 − [MYH9_HUMAN]
Myosin light polypeptide 6 (Fragment) OS = HS GN = MYL6 PE = 1 SV = 1 − [F8VPF3_HUMAN]
Neurobeachin-like protein 2 OS = HS GN = NBEAL2 PE = 1 SV = 2 − [NBEL2_HUMAN]
Nidogen-1 OS = HS GN = NID1 PE = 1 SV = 3 − [NID1_HUMAN]
Epididymal secretory protein E1 (Fragment) OS = HS GN = NPC2 PE = 1 SV = 1 − [G3V2V8_HUMAN]
Puromycin-sensitive aminopeptidase OS = HS GN = NPEPPS PE = 1 SV = 1 − [E9PLK3_HUMAN]
Nuclear transport factor 2 (Fragment) OS = HS GN = NUTF2 PE = 1 SV = 1 − [H3BRV9_HUMAN]
Ubiquitin thioesterase OTUB1 OS = HS GN = OTUB1 PE = 1 SV = 1 − [F5GYN4_HUMAN]
Beta-parvin OS = HS GN = PARVB PE = 1 SV = 1 − [A0A087WZB5_HUMAN]
Pterin-4-alpha-carbinolamine dehydratase OS = HS GN = PCBD1 PE = 1 SV = 2 − [PHS_HUMAN]
Profilin-1 OS = HS GN = PFN1 PE = 1 SV = 2 − [PROF1_HUMAN]
Profilin OS = HS GN = PFN2 PE = 1 SV = 1 − [C9J712_HUMAN]
Glycerol-3-phosphate phosphatase OS = HS GN = PGP PE = 1 SV = 1 − [PGP_HUMAN]
Fibrocystin-L OS = HS GN = PKHD1L1 PE = 2 SV = 2 − [PKHL1_HUMAN]
Periostin OS = HS GN = POSTN PE = 1 SV = 1 − [B1ALD9_HUMAN]
Ribose-phosphate pyrophosphokinase 3 OS = HS GN = PRPS1L1 PE = 1 SV = 1 − [A0A0B4J207_HUMAN]
Serine protease 23 (Fragment) OS = HS GN = PRSS23 PE = 1 SV = 1 − [E9PRR2_HUMAN]
Proteasome subunit alpha type-3 OS = HS GN = PSMA3 PE = 1 SV = 2 − [PSA3_HUMAN]
Proteasome subunit alpha type OS = HS GN = PSMA6 PE = 1 SV = 1 − [G3V295_HUMAN]
Proteasome subunit beta type-2 OS = HS GN = PSMB2 PE = 1 SV = 1 − [PSB2_HUMAN]
26S proteasome non-ATPase regulatory subunit 3 OS = HS GN = PSMD3 PE = 1 SV = 2 − [PSMD3_HUMAN]
26S proteasome non-ATPase regulatory subunit 8 (Fragment) OS = HS GN = PSMD8 PE = 1 SV = 8 − [K7EJR3_HUMAN]
Prostaglandin-H2 D-isomerase OS = HS GN = PTGDS PE = 1 SV = 1 − [PTGDS_HUMAN]
Peroxidasin homolog OS = HS GN = PXDN PE = 1 SV = 2 − [PXDN_HUMAN]
Sulfhydryl oxidase 1 OS = HS GN = QSOX1 PE = 1 SV = 3 − [QSOX1_HUMAN]
Ras-related protein Rab-11A (Fragment) OS = HS GN = RAB11A PE = 4 SV = 1 − [H3BMH2_HUMAN]
Ras-related protein Rab-2B OS = HS GN = RAB2B PE = 1 SV = 1 − [E9PE37_HUMAN]
Ras-related protein Rab-5C (Fragment) OS = HS GN = RAB5C PE = 1 SV = 1 − [F8VVK3_HUMAN]
GTP-binding nuclear protein Ran (Fragment) OS = HS GN = RAN PE = 1 SV = 8 − [F5H018_HUMAN]
Retinoic acid receptor responder protein 2 OS = HS GN = RARRES2 PE = 1 SV = 1 − [RARR2_HUMAN]
60S acidic ribosomal protein P0 (Fragment) OS = HS GN = RPLP0 PE = 1 SV = 1 − [F8VPE8_HUMAN]
40S ribosomal protein S2 (Fragment) OS = HS GN = RPS2 PE = 1 SV = 1 − [H0YEN5_HUMAN]
Ras suppressor protein 1 OS = HS GN = RSU1 PE = 1 SV = 3 − [RSU1_HUMAN]
Syndecan-4 OS = HS GN = SDC4 PE = 1 SV = 2 − [SDC4_HUMAN]
Alpha-1-antichymotrypsin OS = HS GN = SERPINA3 PE = 1 SV = 1 − [G3V3A0_HUMAN]
Plasminogen activator inhibitor 1 OS = HS GN = SERPINE1 PE = 1 SV = 1 − [PAI1_HUMAN]
Glia-derived nexin OS = HS GN = SERPINE2 PE = 1 SV = 1 − [GDN_HUMAN]
SH3 domain-binding glutamic acid-rich-like protein 3 OS = HS GN = SH3BGRL3 PE = 1 SV = 1 − [Q5T123_HUMAN]
Sorbitol dehydrogenase OS = HS GN = SORD PE = 1 SV = 1 − [H0YLA4_HUMAN]
SPARC OS = HS GN = SPARC PE = 1 SV = 1 − [SPRC_HUMAN]
Testican-1 OS = HS GN = SPOCK1 PE = 1 SV = 1 − [TICN1_HUMAN]
Soluble scavenger receptor cysteine-rich domain-containing protein SSC5D OS = HS GN = SSC5D
PE = 1 SV = 3 − [SRCRL_HUMAN]
Stanniocalcin-2 (Fragment) OS = HS GN = STC2 PE = 1 SV = 1 − [H0YB13_HUMAN]
Tissue factor pathway inhibitor 2 OS = HS GN = TFPI2 PE = 1 SV = 1 − [TFPI2_HUMAN]
Thrombospondin-1 OS = HS GN = THBS1 PE = 1 SV = 2 − [TSP1_HUMAN]
Metalloproteinase inhibitor 1 OS = HS GN = TIMP1 PE = 1 SV = 1 − [TIMP1_HUMAN]
Metalloproteinase inhibitor 2 OS = HS GN = TIMP2 PE = 1 SV = 2 − [TIMP2_HUMAN]
Tenascin OS = HS GN = TNC PE = 1 SV = 3 − [TENA_HUMAN]
Tropomyosin alpha-3 chain OS = HS GN = TPM3 PE = 1 SV = 1 − [A0A087WWU8_HUMAN]
Tropomyosin alpha-4 chain OS = HS GN = TPM4 PE = 1 SV = 3 − [TPM4_HUMAN]
Translationally-controlled tumor protein OS = HS GN = TPT1 PE = 1 SV = 1 − [TCTP_HUMAN]
Translin (Fragment) OS = HS GN = TSN PE = 1 SV = 1 − [H7C1D4_HUMAN]
Tubulin beta chain OS = HS GN = TUBB PE = 1 SV = 1 − [Q5JP53_HUMAN]
Polyubiquitin-C (Fragment) OS = HS GN = UBC PE = 1 SV = 1 − [F5GYU3_HUMAN]
Ubiquitin-conjugating enzyme E2 N OS = HS GN = UBE2N PE = 1 SV = 1 − [F8VQQ8_HUMAN]
Versican core protein OS = HS GN = VCAN PE = 1 SV = 3 − [CSPG2_HUMAN]
Vimentin OS = HS GN = VIM PE = 1 SV = 1 − [B0YJC4_HUMAN]
Vacuolar protein sorting-associated protein 29 OS = HS GN = VPS29 PE = 1 SV = 1 − [VPS29_HUMAN]

### Example 6: Concentration, Lyophilization, and Protein Array Studies of Placental ASC

CM from the previous Example was subjected to no treatment (BR), Tangential Flow Filtration through 10 KDa cutoff membrane (TFF; Pall Corporation), or lyophilization (LYP). Tables 5-6 show the concentration data from TFF and LYP.

TABLE 5
Starting sample volumes and concentration factors for TFF.

| | | After | |
|---|---|---|---|
| | Starting | TFF | Concentration |
| Batch | volume | volume | factor |
| Fetal/serum #1 | 90 | 12 | X 7.5 |
| Fetal/serum #2 | 240 | 15 | X 16 |
| Maternal #1 | 220 | 15 | X 15 |
| Maternal #2 | 180 | 10 | X 18 |
| Fetal/SFM #1 | 180 | 12 | X 15 |
| Fetal/SFM #2 | 220 | 18 | X 12 |

TABLE 6
Starting sample protein concentrations
and concentration factors for LYP.

| | | After | |
|---|---|---|---|
| | Original | reconstitution | Concentration |
| Batch | conc. (mg/ml) | conc. (mg/ml) | factor |
| Fetal/serum #1 | 12.4 | 100 | X 8.1 |
| Fetal/serum #2 | 15.6 | 100 | X 6.4 |
| Maternal #1 | 5.74 | 100 | X 17.4 |
| Maternal #2 | 1.7 | 100 | X 58.8 |
| Fetal/SFM #1 | 14.8 | 100 | X 6.8 |
| Fetal/SFM #2 | 20.1 | 100 | X 5 |

Table 7 summarizes expression levels (pg/ml) in the fetal/SF batches with or without concentration processes. +++ indicates values beyond the kit detection limit.

| Protein | BR | TFF | LYP |
|---|---|---|---|
| Collagen 1a | +++ | +++ | +++ |
| IL-1RA (pg/ml) | 142.5 | 78.4 (x1.8) | 38 (x3.75) |
| bFGF (pg/ml) | 189 | 69 (x2.7) | 121.5 (x1.6) |
| Collagen IV a1 | 1250 | 600.5 (x.2.1) | 829.5 (x1.5) |
| (pg/ml) | | | |
| Fibronectin (pg/ml) | +++ | +++ | +++ |
| HGF (pg/ml) | 3434 | 1367.5 (x2.5) | 1722 (x2) |
| MMP-1 (pg/ml) | +++ | +++ | +++ |
| MMP-2 (pg/ml) | 14187 | 1032 (x13.7) | 2016 (x6.7) |
| IL-16 (pg/ml) | 50 | 5 (x10) | 9 (x5.6) |
| VEGF-1 (pg/ml) | 33 | 3 (x11) | 6 (x5.5) |
| IL-4 (pg/ml) | 118.5 | 9.5 (x12.5) | 23 (x5.2) |
| PDGF-AA (pg/ml) | 33 | 28.5 (x1.2) | 18 (x1.8) |
| TIMP1 (pg/ml) | +++ | +++ | +++ |
| TGFb1 (pg/ml) | 746.5 | 405.5 (x1.8) | 130 (x5.7) |

### Example 7: Secretion of Pro-Angiogenic Factors by Placental ASC

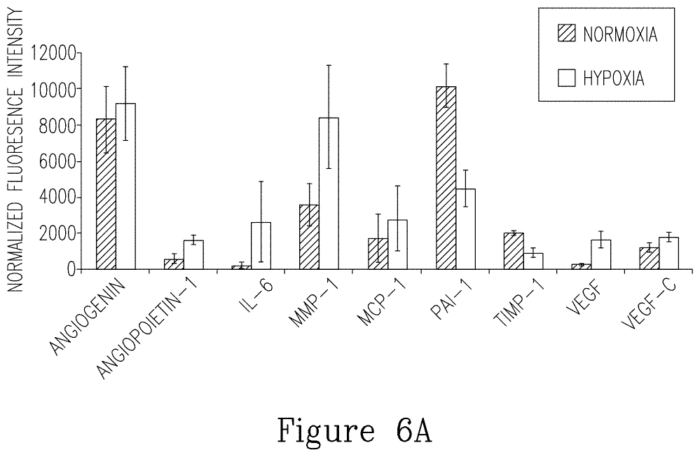

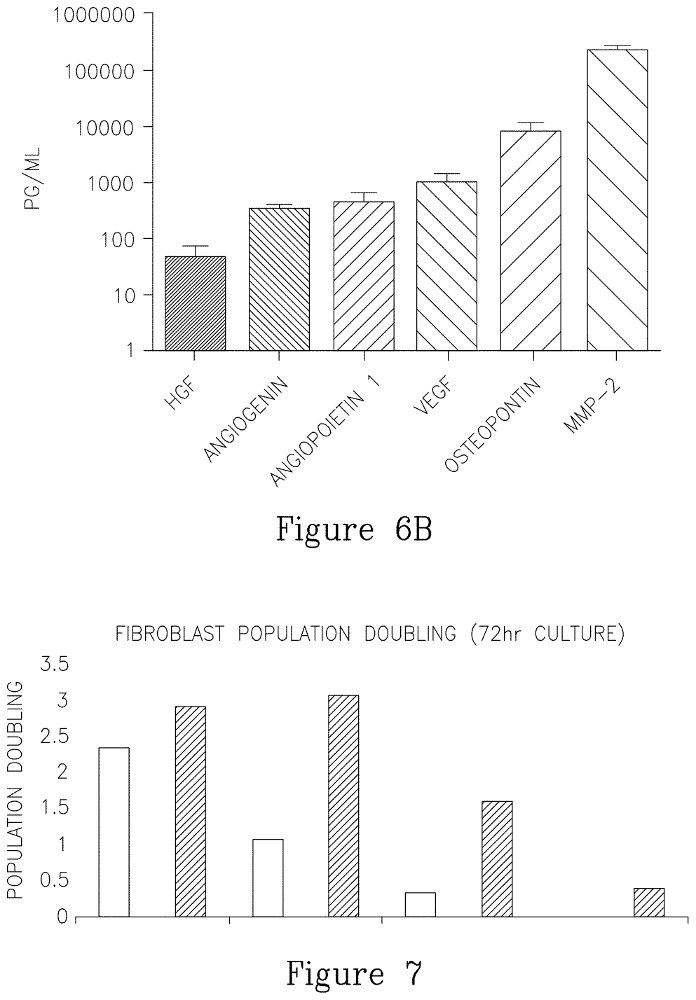

Maternal placental ASC were incubated under normal or hypoxic conditions, and secretion of pro-angiogenic factors was measured by Luminex®. A number of factors were expressed (FIG. 6A). The expression of selected factors was determined by ELISA (FIG. 6B). Thus, placental ASC secrete pro-angiogenic factors.

### Example 8: Placental ASC-CM Increases Proliferation of Dermal Fibroblasts

HDFa (adult, primary human dermal fibroblast; by ATCC cat. #PCS-201-012) cells were expanded in culture and cryopreserved at different stages, namely after 1, 10, and 22 days (2.1, 8.6, or 12.3 population doublings [PD], respectively) in culture, to model young and aged fibroblasts in human dermis. Cells were thawed and incubated for 72 hours in complete fibroblast growth medium (GM; from ATCC) diluted ×2 with either (a) double-distilled water DDW (neg. control); or (b) lyophilized and resuspended (with DDW to 30 mg./ml.) CM from fetal placental ASC (placental ASC-CM). ASC-CM stimulated proliferation of fibroblasts of all ages (FIG. 7).

### Example 9: Placental ASC-CM Protects Dermal Fibroblasts from Oxidative Stress

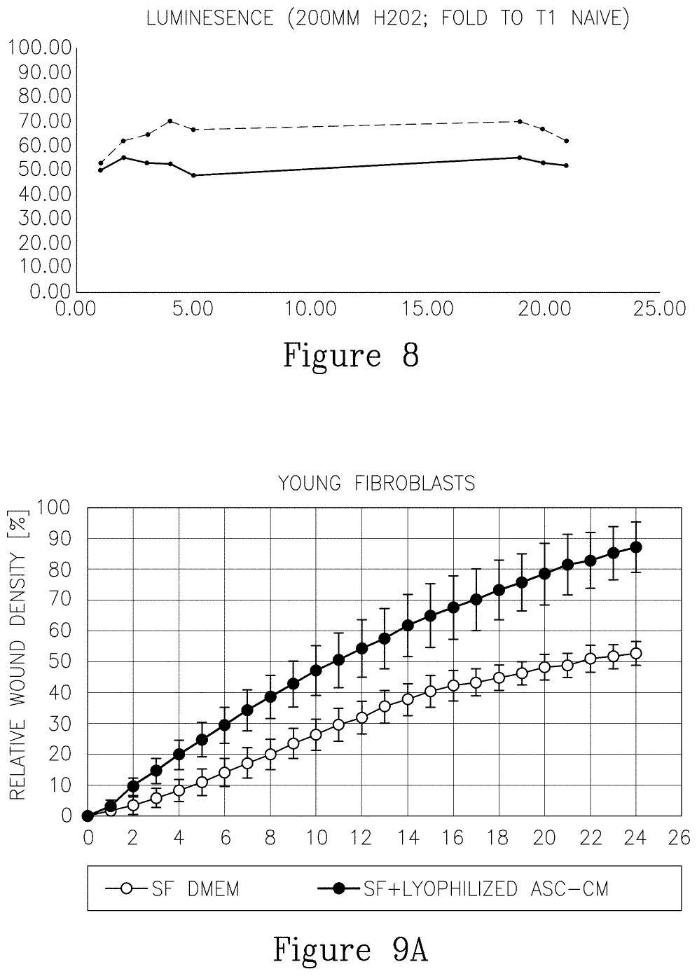

HDFa cells were exposed to 200 micromolar (M) hydrogen peroxide (H₂O₂) for 3 hours, then incubated for 24 hours in expanded in either (a) HDFa complete GM (ATCC cat. #. pcs-201-041) (negative control); or (b) maternal placental ASC-CM produced in HDFa complete GM, after which cell viability was assessed using RealTime-Glo™ MT cell viability assay reagent (Promega). ASC-CM protected the cells from death due to oxidative stress (FIG. 8).

### Example 10: Placental ASC-CM Increases Migration of Dermal Fibroblasts

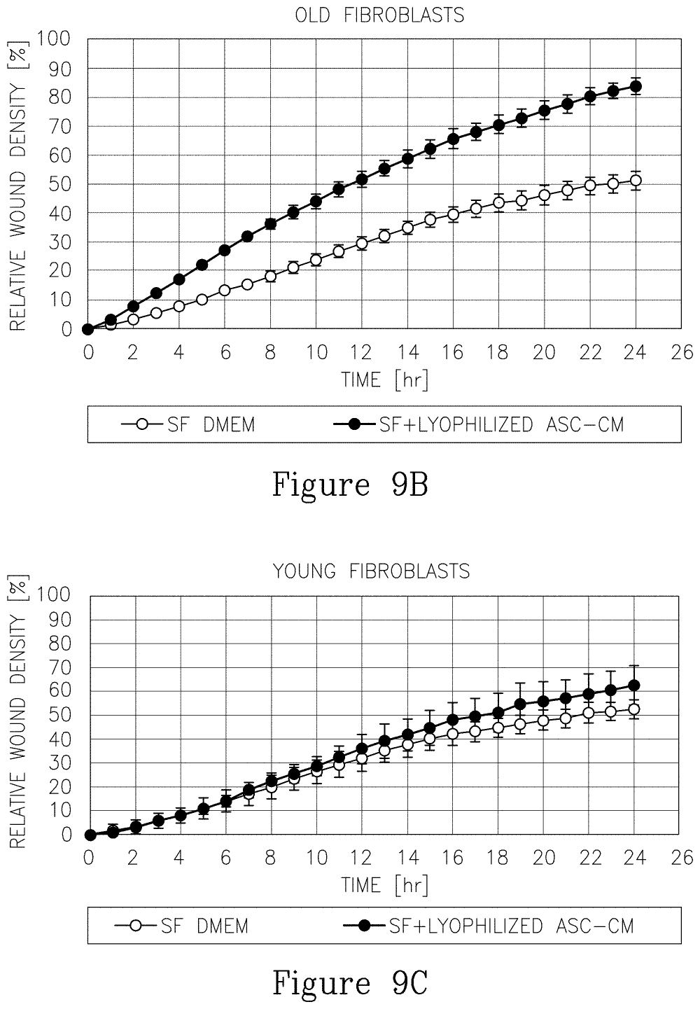

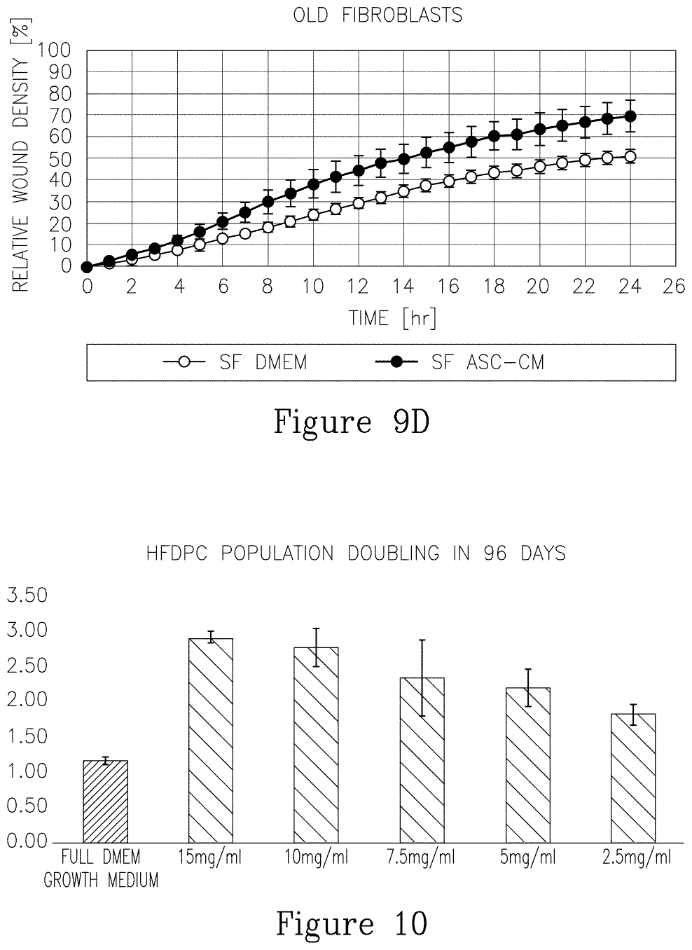

Young or old HDFa cells (0 or 7 PD) were plated in a monolayer and assayed using the IncuCyte® Live-Cell Analysis kit (Essen Bioscience). Monolayers were scratched using WoundMaker™, then incubated, in either (a) serum-free (SF) DMEM (negative control); or (b) fetal placental ASC-CM (from ASC grown in SFM, in a bioreactor) lyophilized and resuspended (5 mg./ml. final concentration) in SF-DMEM. Cell migration into the wound area was assessed using the camera that came with the kit. ASC-CM stimulated migration of both young and old cells, at all timepoints (FIG. 9A-B, respectively). SF-DMEM was also plotted against straight fetal placental ASC-CM (from ASC grown in SFM, in plates). Again, the CM stimulated migration of young and old cells, at all timepoints (FIG. 9C-D, respectively).

### Example 11: Placental ASC-CM Increases Proliferation of Dermal Papilla Cells

Primary Human Follicle Dermal Papilla Cells (HFDPC) were expanded in culture for 96 hours in either complete DMEM growth medium (GM) diluted ×2 with either (a) DDW (negative control); or (b) lyophilized and resuspended (30 mg./ml.) CM from fetal placental ASC (placental ASC-CM). ASC-CM stimulated proliferation of HFDPC (FIG. 10).

### Example 12: Additional Culture Studies of CM from Placental ASC

Fetal and maternal placental ASC-CM are prepared as described in the above Examples and incubated with keratinocytes. Proliferation, migration and growth factor production by the cells are assayed, as described in Madaan A et al., Rajendran R L et al., Hwang I et al., and the references cited therein.

### Example 13: In Vivo Blood Flow Studies of Placental ASC

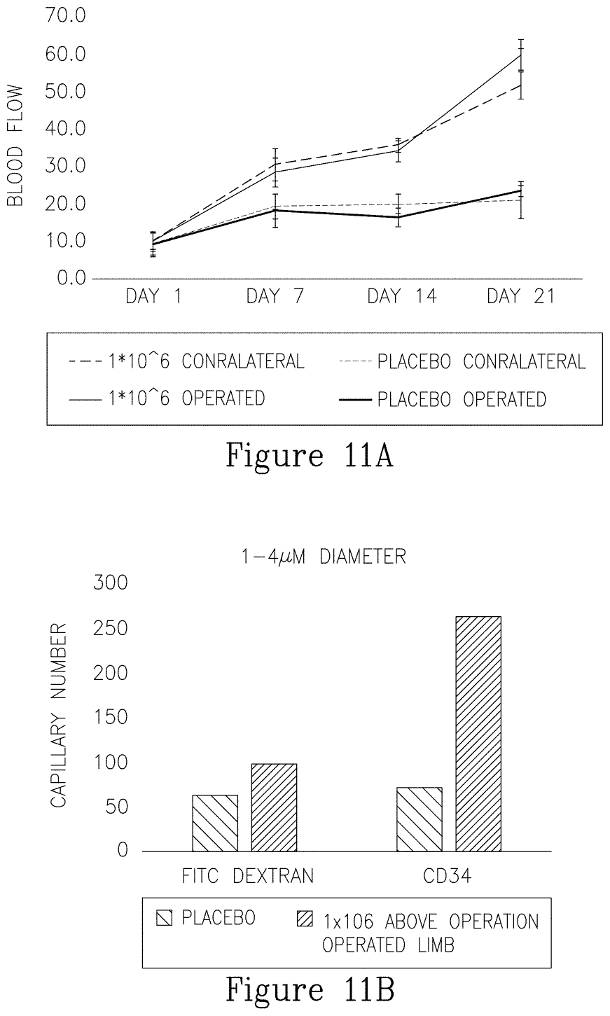

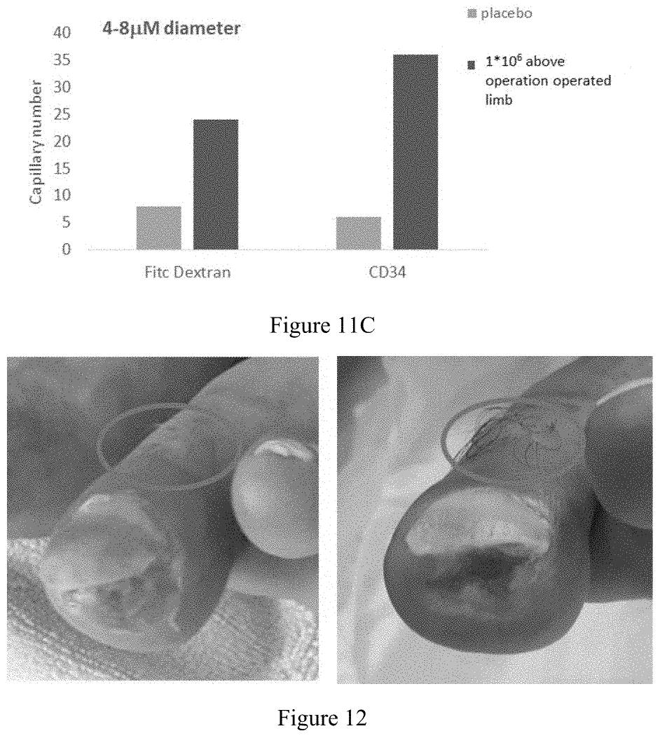

Mice were subjected to femoral artery ligation, and, the next day, were administered one million placental ASC. ASC administration improved blood flow (FIG. 11), which was confirmed by Doppler laser imaging, and formation of functional new blood vessels (FIG. 11-C).

### Example 14: In Vivo Studies of Placental ASC or Factors Derived Therefrom for Hair Regeneration

Human scalp skin grafts are transplanted onto SCID mice as described in Sintov A et al., and treated with vehicle (negative control) vs. placental ASC, ASC lysate, ASC-CM, fractions of the lysate or CM. Histology, Anagen/Telogen ratio (reflective of hair cycle), Ki-67/TUNEL staining (proliferation and apoptosis), hair count, hair diameter, and hair length are performed/analyzed. Enhanced hair growth is indicative of therapeutic efficacy.

### Example 15: Human Testing of Placental ASC for Hair Regeneration

A patient with Buerger's disease and an open, chronic wound that was refractory to treatment was administered 2 doses of 150×10⁶ placental ASC, administered intramuscularly in the affected limb. The wound improved significantly. Additionally, a thick crop of hair sprouted on the dorsal surface of the toes of the affected limb, as shown by a comparison of the affected toe before (FIG. 12A) and after (FIG. 12B) treatment. In other studies, hair growth was repeatedly observed at the site of cell injection.

A placebo-controlled Phase I/II clinical study of hair restoration by injected placental ASC, or topical ASC, is conducted in androgenic alopecia patients. Hair density, diameter, and growth speed are assessed. In other experiments, placental ASC, ASC lysate, ASC-CM, or fractions are similarly assessed.

### Example 16: Human Testing of CM from Placental ASC for Skin Barrier Regeneration

Human subjects are treated immediately following professional facial treatments and chemical peels. One half of the face is treated with a cream containing a basic cream (containing occlusive emollients and/or humectant or reparative moisturizers), while the other half is treated with the basic cream, supplemented with placental ASC-CM. Barrier restoration and skin rejuvenation is assessed several days later by skin care professionals. In other embodiments, placental ASC, lysates, or fractions are utilized.

### Example 17: Human Testing of CM from Placental ASC for Dry Skin Treatment

Human subjects with excessively dry skin are treated in a split-face study, with a basic cream vs. cream supplemented with placental ASC-CM. One month later, skin moisturization is assessed by measuring Trans Epidermal Water Loss (TEWL) and corneometer measurements (Khazaka Electronic, Köln, Germany). In other embodiments, placental ASC, lysates, or fractions are utilized.

### Example 18: Human Testing of CM from Placental ASC for Skin Photodamage Treatment

Human subjects with photodamaged skin are treated in a split-face study, with a basic cream vs. cream supplemented with placental ASC-CM. One month later, skin moisturization is assessed by measuring TEWL and corneometer measurement. In other embodiments, placental ASC, lysates, or fractions are utilized.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace alternatives, modifications and variations that fall within the spirit and broad scope of the claims and description. All publications, patents and patent applications and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the invention.

### REFERENCES (ADDITIONAL REFERENCES MAY BE CITED IN TEXT)

Albertsson P Å. Fractionation of particles and macromolecules in aqueous two-phase systems. Biochem Pharmacol. 1961; 5:351-8. doi: 10.1016/0006-2952(61)90028-4.
Bak D H et al., Human umbilical cord blood mesenchymal stem cells engineered to overexpress growth factors accelerate outcomes in hair growth. Korean J Physiol Pharmacol. 2018 September; 22(5):555-566.
Berthod A, Ruiz-Angel M J, Carda-Broch S. Ionic liquids in separation techniques. J Chromatogr A. 2008; 1184:6-18. doi: 10.1016/j.chroma.2007.11.109.
Bordier C. Phase separation of integral membrane proteins in Triton X-114 solution. J Biol Chem. 1981; 256:1604-7.
Burger D et al, Isolation and Characterization of Circulating Microparticles by Flow Cytometry. Methods Mol Biol. 2017; 1527:271-281. doi: 10.1007/978-1-4939-6625-7_21.
Chase L G et al, Development and Characterization of a Clinically Compliant Xeno-Free Culture Medium in Good Manufacturing Practice for Human Multipotent Mesenchymal Stem Cells. Stem Cells Transl Med. 2012 October; 1(10): 750-758.
Conway Jeannine M. et al, A Flowchart for Selecting an Ointment Base. Am J Pharm Educ. 2014 Feb. 12; 78(1): 16. doi: 10.5688/ajpe78116
Dominici et al, Minimal criteria for defining multipotent mesenchymal stromal cells. The International Society for Cellular Therapy position statement. Cytotherapy. 2006; 8(4):315-7.
Grilo A L, Raquel Aires-Barros M, Azevedo A M. Partitioning in aqueous two-phase systems: fundamentals, applications and trends. Sep Purif Rev. 2016; 45:68-80. doi: 10.1080/15422119.2014.983128.
Hatti-Kaul R. Aqueous two-phase systems. Mol Biotechnol. 2001; 19:269-77. doi: 10.1385/MB:19:3:269.
Hugel B., Martinez M. C., Kunzelmann C., Freyssinet J. M. (2005) Membrane microparticles: Two sides of the coin. Physiology 20, 22-27.
Hwang I et al., Neural Stem Cells Restore Hair Growth Through Activation of the Hair Follicle Niche. Cell Transplant. 2016; 25(8):1439-51.
Kim E S et al., Conditioned Media from Human Umbilical Cord Blood-Derived Mesenchymal Stem Cells Inhibits Melanogenesis by Promoting Proteasomal Degradation of MITF. PLoS One. 2015 May 29; 10(5):e0128078.
Madaan A et al., Review of Hair Follicle Dermal Papilla cells as in vitro screening model for hair growth. Int J Cosmet Sci. 2018 October; 40(5):429-450.
Manjit Jaiswal et al, Nanoemulsion: an advanced mode of drug delivery system. 3 Biotech. 2015 April; 5(2): 123-127.
Jiang B, Li Z-G, Dai J-Y, Zhang D-J, Xiu Z-L. Aqueous two-phase extraction of 2, 3-butanediol from fermentation broths using an ethanol/phosphate system. Process Biochem. 2009; 44:112-7. doi: 10.1016/j.procbio.2008.09.019.
Johansson H-O, Persson J, Tjerneld F. Thermoseparating water/polymer system: A novel one-polymer aqueous two-phase system for protein purification. Biotechnol Bioeng. 1999; 66:247-57. doi: 10.1002/(SICI)1097-0290(1999)66:4<247::AID-BIT6>3.0.CO;2-5. Johnstone R. M., Adam M., Hammond J. R., Orr L., Turbide C. (1987) Vesicle formation during reticulocyte maturation: Association of plasma membrane activities with released vesicles (exosomes). J. Biol. Chem. 262, 9412-9420.
Khan et al (2006), Multiple emulsions: An overview. Current Drug Delivery. 3(4):429-43.
Kinzebach S and Bieback K. Expansion of Mesenchymal Stem/Stromal cells under xenogenic-free culture conditions. Adv Biochem Eng Biotechnol. 2013; 129:33-57.
Liu C, Kamei D T, King J A, Wang D I, Blankschtein D. Separation of proteins and viruses using two-phase aqueous micellar systems. J Chromatogr B. 1998; 711:127-38. doi: 10.1016/S0378-4347(98)00013-9.
Louwrier A. Model phase separations of proteins using aqueous/ethanol components. Biotechnol Tech. 1998; 12:363-5. doi: 10.1023/A:1008818229903.
Lye G J, Asenjo J A, Pyle D L. Extraction of lysozyme and ribonuclease—a using reverse micelles: Limits to protein solubilization. Biotechnol Bioeng. 1995; 47:509-19. doi: 10.1002/bit.260470502.
Mason T G et al (2006), Nanoemulsions: Formation, structure, and physical properties. Journal of Physics: Condensed Matter. 18 (41): R635-R666.
Mimura et al, Growth factor-defined culture medium for human mesenchymal stem cells. Int. J. Dev. Biol. 55: 181-187 (2011).
Pan B. T., Teng K., Wu C., Adam M., Johnstone R. M. (1985) Electron microscopic evidence for externalization of the transferrin receptor in vesicular form in sheep reticulocytes. J. Cell Biol. 101, 942-948.
Parvez S et al., Survey and mechanism of skin depigmenting and lightening agents. Phytother Res. 2006 November; 20(11):921-34.
Rajendran R L et al., Extracellular vesicles derived from MSCs activates dermal papilla cell in vitro and promotes hair follicle conversion from telogen to anagen in mice. Sci Rep. 2017 Nov. 14; 7(1):15560.
Sintov A et al., New topical antiandrogenic formulations can stimulate hair growth in human bald scalp grafted onto mice. Int J Pharm. 2000 Jan. 20; 194(1):125-34.
Sjerobabski-Masnec I, Poduje S. Photoaging. Coll Antropol 2008; 32(2): 177-180.
Van Berlo M, Luyben K C A, van der Wielen L A. Poly (ethylene glycol)-salt aqueous two-phase systems with easily recyclable volatile salts. J Chromatogr B. 1998; 711:61-8. doi: 10.1016/S0378-4347(97)00627-0.
VanWijk M. J., VanBavel E., Sturk A., Nieuwland R. (2003) Microparticles in cardiovascular diseases. Cardiovasc. Res. 59, 277-287.
Xiao J X, Sivars U, Tjerneld F. Phase behavior and protein partitioning in aqueous two-phase systems of cationic-anionic surfactant mixtures. J Chromatogr B. 2000; 743:327-38. doi: 10.1016/S0378-4347(00)00214-0.
Zhang H et al, Identification of distinct nanoparticles and subsets of extracellular vesicles by asymmetric flow field-flow fractionation. Nat Cell Biol. 2018 March; 20(3):332-343. doi: 10.1038/s41556-018-0040-4.

1-39. (canceled)

40. A method for treating, preventing, or ameliorating a skin condition in a subject, comprising administering a composition that comprises a conditioned medium (CM) of a cultured placental adherent stromal cell (ASC), thereby treating, preventing, or ameliorating a skin condition.

41. The method of claim 40, where said skin condition is a compromised skin barrier.

42. The method of claim 41, wherein said compromised skin barrier is a side effect of a facial treatment, laser treatment, micro-needling treatment, chemical peel, or mesotherapy.

43. The method of claim 40, where said skin condition is acne.

44. The method of claim 40, where said skin condition is selected from wrinkling, skin aging, and reduced skin elasticity.

45. The method of claim 40, where said skin condition is a skin laceration.

46. The method of claim 40, where said skin condition is a hyperpigmentation blemish.

47. The method of claim 40, where said skin condition is a hypopigmentation blemish.

48. The method of claim 40, where said skin condition is skin dryness.

49. The method of claim 40, where said skin condition is thinning of epidermis.

50. The method of claim 40, where said skin condition is an elastosis.

51. The method of claim 40, wherein said composition is a cosmetic serum formulation.

52. The method of claim 40, wherein said composition is a foam.

53. The method of claim 40, wherein said composition is a cream.

54. The method of claim 40, wherein said placental ASC have been incubated on a 3D substrate.

55. The method of claim 54, wherein said placental ASC have been incubated in a bioreactor.

56. The method of claim 40, wherein said ASC express a marker selected from the group consisting of CD73, CD90, CD29 and CD105.

57. The method of claim 56, wherein said ASC do not express a marker selected from the group consisting of CD3, CD4, CD11b, CD14, CD19, and CD34.

58. The method of claim 56, wherein said ASC do not express a marker selected from the group consisting of CD3, CD4, CD34, CD39, and CD106.

59. The method of claim 58, wherein more than 50% of said ASC express CD200.
